(19) United States
(12) Patent Application Publication
(10) Pub. No.: US 2026/0294967 A1
(43) Pub. Date: Oct. 1, 2026

(54) TREATMENT AND DIAGNOSIS OF AUTOANTIBODY-MEDIATED EYE DISEASES

(71) Applicant: The Board of Trustees of the University of Illinois, Urbana, IL (US)

(72) Inventor: Sandeep Jain, Chicago, IL (US)

(21) Appl. No.: 19/704,180

(22) Filed: Jun. 10, 2026

Related U.S. Application Data
(63) Continuation of application No. 17/292,094, filed on Nov. 8, 2019.
(60) Provisional application No. 62/757,641, filed on Nov. 8, 2018.
(60) Provisional application No. 62/855,253, filed on May 31, 2019.

(51) Int. Cl. A61K 35/16; A61K 45/06; A61P 27/02

(52) U.S. Cl. CPC A61K 35/16; A61K 45/06; A61P 27/02

(57) ABSTRACT

The present disclosure provides an ophthalmic formulation comprising one or more pharmaceutically acceptable excipients; a pharmaceutically active compound that is capable of reducing the amount or deleterious actions of autoantibodies on the ocular surface, such as IgG; In particular, the present disclosure provides an ophthalmic formulation where the pharmaceutically active compound is capable of treating a clinical condition selected from the group consisting of inflammatory, infectious and immunological ocular surface or intraocular disease that can cause symptoms of ocular discomfort, keratitis, dry eye disease, symblepharon formation, fornix foreshortening, eyelid margin/conjunctival keratinization, subconjunctival fibrosis, retinal gliosis and glaucoma.

### CROSS REFERENCE TO RELATED APPLICATIONS

This application claims the benefit of U.S. Provisional Patent Application No. 62/757,641, filed on Nov. 8, 2018 and U.S. Provisional Patent Application No. 62/855,253, filed on May 31, 2019, the entire contents of which are fully incorporated herein by reference.

### STATEMENT REGARDING FEDERALLY FUNDED RESEARCH

The present disclosure was made with government support under grant number R01 EY024966 awarded by the National Eye Institute (NEI)/National Institutes of Health (NIH). The government has certain rights in the disclosure.

### FIELD OF THE DISCLOSURE

The present disclosure relates to an ophthalmic formulation capable of reducing the concentration or deleterious actions of autoantibodies on the ocular surface and/or inside the eye to prevent or treat inflammatory, immunological, allergic, infectious, and traumatic ocular surface and/or intraocular eye diseases.

### BACKGROUND OF THE DISCLOSURE

Inflammatory, immunological, allergic, infectious, and traumatic ocular surface and intraocular eye diseases can cause various signs and symptoms including, but not limited to, ocular discomfort, ocular redness, a dry eye syndrome, conjunctivitis, keratitis, symblepheron formation, fornix foreshortening, eyelid margin/conjunctival keratinization, and subconjunctival fibrosis. More specific types of ocular surface diseases include ocular graft-versus-host disease (oGVHD), Steven Johnson syndrome, ocular cicatricial pemphigoid (OCP), mild, moderate and severe tear deficient dry eye disease (DED), meibomian gland disease, ocular rosacea, hordeolum, blepharitis, superior limbic keratoconjunctivitis (SLK), tear sufficient DED, floppy eyelid syndrome, neurotrophic eye disease, symptom-sign disconnect (discordant DED), neuropathic pain, thyroid eye disease (Grave's Ophthalmopathy), rheumatoid arthritis-related eye disease, lupus-related eye disease, Sjogren's syndrome, Secondary Sjogren's syndrome, ocular rosacea, allergic keratoconjunctivitis (vernal), viral keratoconjunctivitis (adenovirus EKC, herpesvirus), Thygeson's keratitis, aniridia, keratitis or a postoperative/post-trauma ocular condition, peripheral ulcerative keratitis, keratitis, episcleritis, scleritis, retinal gliosis, Uveitis (anterior or posterior) and glaucoma (open angle or angle closure or normal tension).

Conventional treatments for some of these ocular surface diseases, in particular dry eye syndrome, include (i) instillation of artificial tears for tear supplementation and stimulation and (ii) the use of anti-inflammatory drugs to reduce ocular surface inflammation. Generally, current dry eye treatment involves topical application of artificial tear products/lubricants, tear retention management, stimulation of tear secretion, topical application of antibiotics (e.g., erythromycin or bacitracin ointments), oral administration of tetracyclines (e.g., tetracycline, doxycycline, or minocycline), application of anti-inflammatory compounds and corticosteroids. Conventional treatments for some of intraocular diseases, include (i) treatments to reduce intraocular pressure and (ii) steroid intraocular injections. These treatments are often time consuming, frustrating, and frequently ineffective or variably effective. Moreover, while conventional treatment is effective in reducing symptoms of dry eye syndrome to some extent, it has many undesired side effects, such as burning and stinging sensations. To decrease local side effects and to enhance the patient's comfort and response to treatment is one of the objectives of the present disclosure.

Another shortcoming of conventional treatment in ocular surface disease treatment is inconsistent uptake of active pharmaceutical ingredients into corneal cells and their resident time in the cornea to effectively treat ocular surface disease syndrome without continual application. While ointment or cream formulations may allow longer residence time, such formulations may not disrupt the stratum corneum (superficial cornified layers of skin) and may not reach the blood vessels and nerves that are present deeper in the eyelid tissue.

Yet another problem associated with conventional treatment is that no underlying cause has been directly addressed. Discovering the cause of ocular surface diseases allows more effective treatment. Conventional treatments, for the most part, treat only the symptoms of ocular surface disease.

Accordingly, there is a continuing need for diagnostic kits, compositions and methods for effective treatment of clinical conditions associated with ocular surface and intraocular diseases. In addition, there is a need to address the underlying cause of ocular surface and intraocular disease.

### SUMMARY OF THE DISCLOSURE

Some aspects of the disclosure provide an ophthalmic formulation comprising: (a) one or more pharmaceutically acceptable ophthalmic excipients; and (b) an immunoglobulin G (IgG) or a fragment thereof.

Some aspects of the disclosure provide an ophthalmic formulation comprising: (a) one or more pharmaceutically acceptable ophthalmic excipients; and (b) pooled plasma derived pooled human immune globulin G (IgG).

In addition, the disclosure provides an ophthalmic formulation comprises: (a) one or more pharmaceutically acceptable ophthalmic excipients; and (b) an ocularly pharmaceutically active compound for treatment of a clinical condition selected from the group consisting of inflammatory, infectious, immunological, allergic, and/or traumatic ocular surface disease or intraocular disease, wherein the pharmaceutically active compound comprises IgG.

In various aspects, the disclosure provides an ophthalmic formulation comprising a pharmaceutically active compound that is capable of reducing the amount or deleterious biological effects of autoantibodies over the ocular surface or inside the eye, such autoantibodies being generated in response to a citrullinated-protein (referred to herein as “ACPA autoantibodies” which are generated in response to citrullination of proteins) or an autoantibody generated in response to a homocitrullinated-protein (referred to herein as “anti-CarP antibodies” which are generated in response to carbamylation of proteins)—or native autoantibodies generated in response to native proteins. Citrullinatoin and carbamylation are two types of post-translational modification (PTM) of proteins. In related aspects, the pharmaceutically active compound comprises immunoglobulin G (IgG) or a fragment thereof. These ophthalmic formulations also may comprise one or more pharmaceutically acceptable ophthalmic excipients.

In various aspects, the disclosed ophthalmic formulations comprise one or more of pharmaceutically acceptable ophthalmic excipients. For example, the pharmaceutically acceptable ophthalmic excipients is selected from Cyclodextrins, Carbopol or carbomer or acrylic acid polymers, Poloxamers, Xyloglucan, Methylcellulose, Hydroxypropyl Methylcellulose, Ethyl (Hydroxyethyl) Cellulose, Pseudolatexes, Cellulose Acetate Phthalate, Gellan Gum, Alginate, Carrageenans, Hyaluronic Acid, Sodium acetate, Edetate disodium, Hypromellose, Acetic acid, Alcohol, Alginic acid, Amerchol-cab, Antipyrine, Benzalkonium chloride, Benzododecinium bromide, Boric acid, Caffeine, Calcium chloride, Carbomer 1342, Carbomer 934P, Carbomer 940, Carbomer homopolymer type B (allyl pentaerythritol cross-linked), Carboxymethylcellulose sodium, Castor oil, Cetyl alcohol, Chlorobutanol, Citric acid, Citric acid monohydrate, Creatinine, Divinylbenzene styrene copolymer, Ethylene vinyl acetate copolymer, Gellan gum (low acyl), Glycerin, Glyceryl stearate, Hypromelloses, Lanolin, Lauralkonium chloride, Lauroyl sarcosine, Magnesium chloride, Methylparaben, Mineral oil, Nonoxynol-9, Octoxynol-40, Petrolatum, Phenylethyl alcohol, Phenylmercuric acetate, Phenylmercuric nitrate, Polidronium chloride, Poloxamer 188 or 407, Polycarbophil, Polyethylene glycol 400 or 8000, Polyoxyl 35 castor oil, Polyoxyl 40 hydrogenated castor oil, Polyoxyl 40 stearate, Polypropylene glycol, Polysorbate 20, Polyvinyl alcohol, Potassium chloride, Potassium sorbate, Povidone K29/32, Povidone K30, Povidone K90, Povidones, Propylene glycol, Propylparaben, Soda ash, Sodium acetate, Sodium bisulfate, Sodium borate, Sodium borate decahydrate, Sodium carbonate, Sodium chloride, Sodium citrate, Sodium metabisulfite, Sodium nitrate, Sodium sulfate, Sodium sulfite, Sodium thiosulfate, Sorbic acid, Sorbitol, Stabilized oxychloro complex, Sulfuric acid, Thimerosal, Titanium dioxide, Tocophersolan, Trisodium citrate dehydrate, Tromethamine, Tyloxapol, Xanthan gum, Zinc chloride, or a combination thereof.

Still another aspect of the disclosure provides an ophthalmic formulation comprising (a) a pharmaceutically acceptable ophthalmic excipient; (b) a therapeutically effective amount of a pharmaceutically active compound comprising pooled human immunoglobulin G (IgG); and/or (c) a therapeutically effective amount of pooled human plasma proteins, pooled human plasma lipids or a combination thereof. As used herein, the term “therapeutically effective” includes having an immunomodulatory effect or the ability to neutralize autoantibodies.

In any of the disclosed ophthalmic formulations, the pharmaceutically active compound may comprise autologous IgG purified from autologous plasma/serum; a multimerized IgG1 Fc molecule, an IgG1 Fc hexamer; an IgG2a Fc multimers, stradomers; multivalent Fc structures; glycoengineered sialylated IgG; IgG-Fc Glycosylation; synthetic IgG and fragments thereof, or a combination thereof.

In various aspects, any of the disclosed ophthalmic formulation may further comprise a second pharmaceutically active compound selected from a steroid, an anti-inflammatory agent, such as methylprednisone, prednisone, dexamethasone, cyclosporine, Lifitegrast®, a non-steroidal anti-inflammatory drug, a mucolytic agent, such as N-acetylcysteine, Nacystelyn, N-acetylglucosamine), a PAD enzyme inhibitor, such as paclitaxel, glucocorticoids or CI-amidine, or NETs dismantling agent (DNase or Heparin), Targeted Fab antibody fragments, Fc receptor blocking peptides, Fc receptor blocking antibodies, recombinant peptide containing pathogenic epitopes, conventional synthetic DMARDs (Methotrexate, Leflunomide/Teriflunomide, Sulfasalazine, Chloroquine/Hydroxychloroquine), TNF-α targeted therapy (Infliximab, Adalimumab, Etanercept, Golimumab, Certolizumab pegol), B-cell targeted therapy (Rituximab, Ofatumumab, Belimumab, Atacicept, Tabalumab), T-cell targeted therapy (Abatacept, Belatacept), Interleukin Targeted therapy (Tocilizumab, Anakinra, Canakinumab, Rilonacept, Secukinumab), Growth and differentiation factors (Denosumab, Mavrilimumab), JAK pathway inhibitors (Tofacitinib, Baricitinib, Filgotinib), and a combination thereof.

In some embodiments, the concentration or amount of IgG present in said ophthalmic formulation ranges from about 0.01 mg/mL by weight to about 1 g/mL (1000 mg/mL) by weight, for example or from about 0.05 mg/ml by weight to about 1 g/ml by weight, or from about 0.1 mg/ml by weight to about 1 g/ml or from about 0.1 mg/ml by weight to about 0.5 mg/ml or 0.05 mg/ml by weight to about 0.5 mg/ml by weight or 0.01 mg/ml by weight to about 0.1 mg/ml by weight. In certain embodiments the concentration or amount of IgG may be about 0.01 mg/ml, in other embodiments the concentration or amount of IgG may be about 0.05 mg/mL. In certain embodiments the concentration or amount of IgG may be about 1 mg/mL, in other embodiments the concentration or amount of IgG may be about 10 mg/mL.

In various embodiments, the concentration or amount of IgG may be about 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.15 mg/mL, 0.2 mg/mL, 0.25 mg/mL, 0.3 mg/mL, 0.35 mg/mL, 0.4 mg/mL, 0.45 mg/mL, 0.5 mg/mL, 0.55 mg/mL, 0.6 mg/mL, 0.65 mg/mL, 0.7 mg/mL, 0.75 mg/mL, 0.8 mg/mL, 0.85 mg/mL, 0.9 mg/mL, 0.95 mg/mL, 1.0 mg/mL, 1.5 mg/mL, 2.0 mg/mL, 2.5 mg/ml, 3.0 mg/ml, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, 650 mg/mL, 700 mg/mL, 750 mg/mL, 800 mg/mL, 850 mg/mL, 900 mg/mL, 950 mg/mL, or 1000 mg/mL.

In various exemplary embodiments, the amount of IgG is about 10 mg/ml or less. In certain embodiments the ophthalmic formulation can include a concentration or amount of IgG is about 0.1 mg/ml or less, about 0.15 mg/mL or less, 0.2 mg/mL, about 0.25 mg/ml or less, about 0.3 mg/ml or less, about 0.35 mg/mL or less, about 0.4 mg/ml or less, about 0.45 mg/ml or less, about 0.5 mg/ml or less, about 0.55 mg/ml or less, about 0.6 mg/mL or less, about 0.65 mg/ml or less, about 0.7 mg/ml or less, about 0.75 mg/mL or less, about 0.8 mg/ml or less, about 0.85 mg/ml or less, about, 0.9 mg/mL or less, about 0.95 mg/ml or less, about or 1.0 mg/mL or less.

The term “pooled plasma-derived pooled human immunoglobulin G” refers unfractionated plasma pooled from several donors followed by fractionation to separate immunoglobulin G. The present disclosure provides for ophthalmic compositions comprising plasma pooled from several donors which is dispensed unfractionated in single use droppers or multidose bottles in various dilutions based on IgG concentration (0.01% to 10%). The pooling of plasma can also be done prior to processing steps. This pooled plasma & immunoglobulin (PPIG) preparation differs from conventional plasma products (e.g. PRP) in that conventional plasma products are prepared from blood of one subject whereas PPIG preparation is made from pooled plasma of several hundreds to thousands of healthy subjects. The final product comprises pooled IgG and plasma proteins, with or without platelets and platelet activation products. The pooled plasma derived IgG may also comprise plasma lipids.

In yet another iteration, pooled plasma-derived pooled human immunoglobulin G (0.01% to 10% concentration) from several hundreds to thousands of healthy subjects is formulated in plasma or serum (allogeneic or autologous) with ophthalmic excipient(s). This ophthalmic formulation will provide a combination of autoantibody neutralizing pooled IgG and ocular surface regeneration promoting proteins, cytokines and growth factors of serum/plasma.

In various aspects, the IgG present in any of the disclosed ophthalmic formulations comprises serum/plasma-derived pooled immunoglobulin G (OSIG: ocular surface immune globulin), Multimerized IgG1 Fc molecule (IgG1 Fc hexamer), IgG2a Fc multimers (stradomers), Multivalent Fc structures, or a combination thereof.

In various aspects, the IgG present in any of the disclosed ophthalmic formulations is an antigen binding fragment in which one or more antibody domains are truncated or absent, genetically-engineered antibodies or protein binding fragments thereof, single chain antibodies or antibodies that can bind to more than one epitope or antibodies that can bind to one or more different antigens. For example, the fragment of IgG is s an antigen binding fragment, single chain antibodies, Fv fragment, Fab fragment, Fab′ fragment, or F (ab) 2 fragment.

In various aspects, the IgG present in any of the disclosed ophthalmic formulation comprises IgG1, IgG2, IgG3, and IgG4 or a combination thereof. Additionally or alternatively, the IgG can include or be derived from any suitable source such as stem cell preparations containing IgG, manufactured IgG or IgG stem cells, glycoengineered sialylated IgG, IgG-Fc Glycosylation or the like, or a combination thereof.

In various aspects, the IgG present in any of the disclosed ophthalmic formulations comprises a preselected concentration of autologous IgG purified from autologous plasma/serum using a separation process such as affinity chromatography using Protein A beads, or another IgG separation process.

In various aspects, the IgG present in any of the disclosed ophthalmic formulation neutralizes an autoimmune antibody or an antibody that specifically binds to a citrullated protein.

In various aspects, the pH of any of the disclosed ophthalmic formulations is about pH 6 to about pH 8 or about pH 6.2 to about pH 7.2, about pH 6.4 to about pH 7.4, or about pH 6.5 to about pH 7.5, about pH 6.6 to about pH 7.6, about pH 6.8 to about pH 7.8. For example, the pH is about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 7.6, or about 7.7, or about 7.8, or about 7.9, or about 8.0.

In various aspects, any of the disclosed ophthalmic formulations comprise on one or more pharmaceutically acceptable excipient comprises polyvinyl alcohol, povidone, hydroxypropyl methyl cellulose, poloxamers, polyols, Carbopol, pluronics, carbomers, carboxymethyl cellulose, hydroxyethyl cellulose, cyclodextrins, phosphate buffer, citrate buffer, Tris buffer, sodium chloride, potassium chloride, polysorbate 80, vegetable oil, preservative or a combination thereof.

Any of the disclosed ophthalmic formulations may be used to treat an inflammatory, infectious or immune disease at the mucosa, or to exhibit an immunomodulatory effect at the mucosa. The ophthalmic formulations may treat an inflammatory, infectious or immune disease or have an immunomodulatory effect at the mucosa in the in the oral cavity, nasal cavity, bladder, tracheobronchial passages, ear canal and cavity, synovial (joint) cavity, vaginal cavity or over the skin.

In various aspects, the disclosed ophthalmic formulation comprises multi-dose vials with preservative or a single dose sterile container without preservative.

In various aspects, the disclosure provides a method of treating a clinical condition in a patient a need, comprising a step of administering to the patient an ophthalmic formulation as disclosed herein, where the clinical condition is a an inflammatory ocular surface disease or intraocular eye diseases, an infectious ocular surface disease or intraocular eye diseases and/or an immunological ocular surface disease or intraocular eye disease. In various aspects, the disclosure provide a method treating an inflammatory, infectious and/or immunological ocular disease in a patient in need, comprising a step of administering to a patient an ophthalmic formulation as disclosed herein.

In various aspects, the disclosure provides a method of reducing, relieving or preventing ocular discomfort in a patient suffering from a clinical condition, comprising a step of administering to the patient an ophthalmic formulation disclosed herein, where the clinical condition is a an inflammatory ocular surface disease or intraocular eye diseases, an infectious ocular surface disease or intraocular eye diseases and/or an immunological ocular surface disease or intraocular eye disease. In any of these methods, the ocular discomfort comprises one or more of a foreign body sensation, light sensitivity, stinging, irritation, soreness, dryness, burning, redness, itching or scratchiness.

The disclosure provides a method of treating a clinical condition in a patient in need, comprising a step of administering to the patient an ophthalmic formulation as disclosed herein, where the clinical condition is an inflammatory, infectious and/or immune disease at the mucosa in the oral cavity, nasal cavity, bladder, tracheobronchial passages, ear canal and cavity, synovial (joint) cavity, vaginal cavity or over the skin.

The disclosure also provides a method of inducing an immunomodulatory effect at the mucosa in a patient in need, comprising a step of administering to the patient an ophthalmic formulation as disclosed herein, wherein the mucosa is in the oral cavity, nasal cavity, bladder, tracheobronchial passages, ear canal and cavity, synovial (joint) cavity, vaginal cavity or over the skin.

The disclosure also provides for use of any of the ophthalmic formulations for the preparation of a medicament for the treatment of a clinical condition in a patient a need, where the clinical condition is an inflammatory ocular surface disease or intraocular eye diseases, an infectious ocular surface disease or intraocular eye diseases and/or an immunological ocular surface disease or intraocular eye disease. In various aspects, the disclosure provides for use of any of the ophthalmic formulations for the preparation of a medicament for reducing, relieving or preventing ocular discomfort in a patient suffering from a clinical condition, wherein the clinical condition is a an inflammatory ocular surface disease or intraocular eye diseases, an infectious ocular surface disease or intraocular eye diseases and/or an immunological ocular surface disease or intraocular eye disease. In any of these uses, the ocular discomfort comprises one or more of a foreign body sensation, pain, light sensitivity, stinging, irritation, soreness, dryness, burning, redness, itching or scratchiness.

The disclosure also provides for use of an ophthalmic formulation disclosed herein for the preparation of a medicament for the treatment of a clinical condition in a patient in need, wherein the clinical condition is an inflammatory, infectious and/or immune disease at the mucosa in the oral cavity, nasal cavity, bladder, tracheobronchial passages, ear canal and cavity, synovial (joint) cavity, vaginal cavity or over the skin. In addition, the disclosure provide for use of an ophthalmic formulation disclosed herein for the preparation of a medicament for inducing an immunomodulatory effect at the mucosa in a patient in need, wherein the mucosa is in the oral cavity, nasal cavity, bladder, tracheobronchial passages, ear canal and cavity, synovial (joint) cavity, vaginal cavity or over the skin.

The disclosure also provides compositions comprising any of the ophthalmic formulations described herein for use in treating a clinical condition. In various aspects, the compositions may be used for treating an inflammatory, infectious and/or immunological ocular disease in a patient in need. In various aspects, compositions comprising any of the ophthalmic formulations described herein may be used for reducing, relieving or preventing ocular discomfort in a patient suffering from a clinical condition, wherein the clinical condition is a an inflammatory ocular surface disease or intraocular eye diseases, an infectious ocular surface disease or intraocular eye diseases and/or an immunological ocular surface disease or intraocular eye disease. In related aspects, the ocular discomfort comprises one or more of a foreign body sensation, pain, light sensitivity, stinging, irritation, soreness, burning, dryness, redness, itching or scratchiness.

The disclosure provides for a composition for treating a clinical condition in a patient in need, wherein the composition comprises an ophthalmic formulation as disclosed herein, wherein the clinical condition is an inflammatory, infectious and/or immune disease at the mucosa in the oral cavity, nasal cavity, bladder, tracheobronchial passages, ear canal and cavity, synovial (joint) cavity, vaginal cavity or over the skin. In addition, the disclosure provides for a composition for inducing an immunomodulatory effect at the mucosa in a patient in need, wherein the composition comprises an ophthalmic formulation as disclosed herein, wherein the mucosa is in the oral cavity, nasal cavity, bladder, tracheobronchial passages, ear canal and cavity, synovial (joint) cavity, vaginal cavity or over the skin.

Exemplary clinical conditions treatable according to the exemplary methods, use and compositions herein include, but are not limited to, immune eye disease, metabolic eye disease, allergic eye disease, traumatic eye disease, infectious eye disease and genetic eye disease, such as, but not limited to, ocular graft-versus-host disease (oGVHD), Steven Johnson syndrome, ocular cicatricial pemphigoid (OCP), mild, moderate and severe tear deficient dry eye disease (DED), meibomian gland disease, hordeolum, ocular rosacea, blepharitis, superior limbic keratoconjunctivitis (SLK), tear sufficient DED, floppy eyelid syndrome, neurotrophic eye disease, symptom-sign disconnect (discordant DED), neuropathic pain, thyroid eye disease (Grave's Ophthalmopathy), rheumatoid arthritis-related eye disease, lupus-related eye disease, Sjogren's syndrome, Secondary Sjogren's syndrome, ocular rosacea, allergic keratoconjunctivitis (vernal), viral keratoconjunctivitis (adenovirus EKC, herpesvirus), Thygeson's keratitis, retinal gliosis, viral keratoconjunctivitis (adenovirus EKC), Thygeson's keratitis, aniridia, keratitis or a postoperative/post-trauma ocular condition, peripheral ulcerative keratitis, keratitis, episcleritis, scleritis, Uveitis (anterior or posterior), retinitis, and glaucoma (open angle or narrow angle or normal tension). For example, the exemplary methods herein can be used to treat disease from arising from various conditions, such as keratitis due to sterile inflammation (e.g. PUK) or due to a viral (e.g. adenoviral subepithelial infiltrates, herpesvirus dendritic epithelial ulcer), bacterial (e.g. Staphylococcus, Pseudomonas) or fungal (e.g. Candida, Acanthamoeba) infection. Additionally, the exemplary methods herein can be used to treat postoperative/post-trauma ocular condition or an ocular condition associated with post-ocular surface reconstruction surgery, antimetabolite application to eye surface, pterygium surgery, glaucoma surgery, cataract surgery, refractive surgery (LASIK, LASEK or PRK), keratoprosthesis surgery, vitreoretinal surgery or radiation or chemical (alkali or acidic) or traumatic injury.

In various aspects, the patient has autoantibodies present in a biological sample. In related aspects, the autoantibodies present in the biological sample are anti-citrullinated protein antibodies. In certain aspects, the biological sample is ocular fluid. In various aspects, the ocular fluid is tear fluid, eye wash fluid, anterior aqueous humor or posterior vitreous humor.

The disclosure provides for methods for treating an ocular disease comprising the steps of administering a therapeutically effective amount of therapeutic amount of allogeneic or autologous IgG ophthalmic formulation to a patient in need of such a treatment. The disclosure also provides for the use of therapeutic amount of allogeneic or autologous IgG ophthalmic formulation for the preparation of a medicament for treatment of an ocular disease in a patient in need of such a treatment. In addition, the disclosure provides for a composition for treating an ocular disease comprising a therapeutically effective amount of therapeutic amount of allogeneic or autologous IgG ophthalmic formulation to a patient in need of such a treatment.

In any of the disclosed methods, uses or compositions, the ophthalmic formulation is administered as an eye drop formulation, topical liquid, gel formulation, emulsion formulation, suspension formulation, ointment formulation or injectable formulation. The ophthalmic formulation, medicament or composition may be administered at least once a day to a subject. In various embodiments, the IgG ophthalmic formulation is administered once, twice, three, four, five, six, seven, eight, nine or ten times a day to a subject. In an embodiment, the IgG ophthalmic formulation is administered at least once every one to three weeks to a subject. In various embodiments, one of more does of IgG ophthalmic formulation is administered weekly, biweekly, monthly or bimonthly. In various aspects, the concentration of IgG is defined by a 5%, 10% or 20% ocular surface immunoglobulin (OSIG) solution. In various embodiments, the concentration of IgG administered is 5%, 6%, 7%, 8%, 9%, 10%, 11%, 12%, 13%, 14%, 15%, 16%, 17%, 18%, 19%, or 20% OSIG solution. In yet another embodiment, the IgG ophthalmic formulation is administered inside the eye as an intraocular injection. In an embodiment, the IgG ophthalmic formulation is administered to the ocular surface as eye drops or ointment.

In certain embodiments, pooled IgG ophthalmic formulation (modified or unmodified) is manufactured using blood from human subjects and then is administered in a specific concentration to the ocular surface or inside the eye of the same subject. To enable delivery of OSIG across tissues, subconjunctival or subtenon injection is administered or OSIG preparation may be modified (using nanotechnology such as polymeric/biological spheres, particles or membranes (with or without surface modifications). Other methods to enhance penetration of OSIG include using permeants such as benzalkonium, charge based strategies such as iontophoresis or increasing residence time over the ocular surface such as by enhancing viscosity or adsorption in contact lenses or punctal plugs. OSIG eye drops can be formulated as Microemulsions. The time period of contact with cornea and bioavailability of OSIG eye drops may be increased by enhancing viscosity by using hydrophilic polymers of high molecular weight which do not diffuse through biological membranes and which form three-dimensional networks in the water. Examples of such polymers include polyvinyl alcohol, poloxamers, hyaluronic acid, carbomers, carbopol, and polysaccharides, that is, cellulose derivatives, gellan gum, and xanthan gum. The corneal and intraocular penetration of OSIG eye drops can be enhanced by modifying the continuity of corneal epithelium structure using chelating agents, preservatives (like benzalkonium chloride), surfactants, and bile acid salts or by using Cyclodextrin complexes. For intraocular delivery or delivery to corneal, subconjunctival or lacrimal gland, Polymeric, solid, multicompartment drug delivery systems of OSIG are used.

Nanoparticles are polymeric carriers, built from biodegradable, biocompatible, natural, or synthetic polymers with often mucoadhesive properties. Ingredients used in its development, for the purpose of application to the eyeball, are poly(alkyl cyanoacrylate), polylactic acid, poly(epsilon-caprolactone), poly(lactic-co-glycolic acid), chitosan, gelatin, sodium alginate, and albumin. These forms can be divided into nanospheres, the solid, monolithic spheres built from dense polymer matrix, in which the OSIG is scattered, and nanocapsules constituting reservoirs, built from polymer membrane surrounding OSIG in solid or liquid form. Liposomes are also used to deliver OSIG across eye tissues. Liposomes are phospholipid drug carriers usually built of phosphatidylcholine, stearylamine, and various amounts of cholesterol or lecithin and α-L-dipalmitoyl-phosphatidylcholine. Niosomes, Discosomes and Dendrimers are also used to deliver OSIG across eye tissues.

In another embodiment, the disclosure provides for a method of detecting the presence of autoantibodies in a subject, the method comprises detecting the level of autoantibodies in a sample of ocular fluid obtained from the subject, where the subject is suffering from or at risk of developing an ocular surface disorder. In related aspects, the method further comprises a step of comparing the level of autoantibodies in the sample obtained from the subject with a control level of autoantibodies. For example, the ocular fluid is tear fluid, eye wash, anterior chamber aqueous humor or posterior chamber vitreous humor.

The disclosure also provides for method of diagnosing, determining the risk of developing or monitoring of an ocular surface disorder in a subject, the method comprises detecting the level of autoantibodies in a sample obtained from the subject and comparing the level of antibodies in the sample obtained from the subject with a control level of said autoantibodies, where the control level of autoantibodies is used to diagnose, determine the risk of developing or monitor ocular surface disorder in the subject. In related aspects, the autoantibodies are native autoantibodies, anti-CarP autoantibodies or ACPA antibodies. In various aspects, the autoantibody comprises a plurality of autoantibodies. In related aspects, the antigen detecting the autoantibodies comprises citrullinated proteins, citrullinated peptide sequences or a combination thereof. In related aspects, the antigen detecting the autoantibodies comprises carbamylated proteins, carbamylated peptide sequences or a combination thereof.

In any of the disclosed methods the sample is ocular fluid, wherein ocular fluid is tear fluid, eye wash, anterior chamber aqueous humor or posterior chamber vitreous humor.

In any of the disclosed methods, the level of autoantibodies is detected by an enzymatic method, a spectrometric method, a chromatographic method, an immunological method, or a combination thereof. In an exemplary embodiment, a method can include a step of determining the progression of the ocular surface disorder or determining efficacy of a therapy in a subject suffering from, suspected of suffering from, or of being predisposed to, an ocular surface disorder. In any of the disclosed methods, the control level of autoantibodies comprises the level of said autoantibody in the subject prior to commencement of a therapy, the level of said autoantibody in the subject at an earlier stage of a therapy, or a combination thereof.

The disclosure also provides for a diagnostic kit comprising an array of antigen panels for detecting autoantibodies comprising of citrullinated proteins, citrullinated peptide sequences or a combination thereof. The diagnostic kit may be used for carrying out any of the methods described herein.

In an exemplary embodiment, an Fc receptor blocking composition can be formulated for ocular applications. For example, the Fc receptor blocking composition can be further defined by an Fc receptor blocking ocular peptide concentration and optionally, a suitable carrier. In another embodiment, the Fc receptor blocking composition can be further defined by an Fc receptor blocking ocular antibody concentration and optionally, a suitable carrier. In addition, the Fc blocking compositions may be used to treat an inflammatory, infectious or immune disease at the mucosa, or to exhibit an immunomodulatory effect at the mucosa. The Fc blocking compositions may treat an inflammatory, infectious or immune disease or have an immunomodulatory effect at the mucosa in the in the oral cavity, nasal cavity, bladder, tracheobronchial passages, ear canal and cavity, synovial (joint) cavity, vaginal cavity or over the skin.

The disclosure also provides for a concentrated ocular or mucosal site IgG composition. In accordance with the principles herein a concentrated ocular IgG composition is set forth. The composition can further include an anti-inflammatory agent.

The disclosure also provides for a therapeutic dosage for the treatment of an ocular immune disease, where the ocular immune disease is indicated by levels of anti-citrullinated protein autoantibodies determined from ocular fluid removed from a treatment site, and the therapeutic dosage can be formulated using a concentrated ocular IgG composition. In certain embodiments the therapeutic dosage can be determined based on tears removed from a subject, where the subject is suffering from an immune disease, such as dry eye disease. In certain embodiments, a therapeutic dosage can include a composition further defined by an concentrated ocular IgG composition in a concentration to reduce ACPA antibodies concentrated amounts and/or effects via application at a treatment site. For example, the therapeutic dosage can include a concentrated ocular IgG composition ranging from about 0.01 mg/mL by weight to about 1 g/mL by weight.

In some embodiments, a diagnostic kit includes a testing device for determining a concentration of anti-citrullinated protein autoantibodies in an ocular fluid, such as tears; and a dosing device for indicating a therapeutic treatment dosage and regime sufficient to treat and/or reduce damaging effects of the anti-citrullinated protein autoantibodies concentration in the ocular fluid. In related aspects, the ocular fluid is tear fluid, eye wash, anterior aqueous humor or posterior vitreous humor.

In an exemplary embodiment, a method of fabricating a treatment dosage for an immune injury detected in ocular fluid removed from a treatment site comprising providing a concentration of IgG configured to reduce levels and/or effects of anti-citrullinated protein autoantibodies when administered to the treatment site; and adding a carrier to the concentration of IgG thereby forming a treatment dosage configured to enable delivery of the IgG to the treatment site.

In another exemplary embodiment, a method of fabricating a treatment dosage can include providing a concentration of IgG within a range of about 0.01% to about 20% ocular surface immunoglobulin (OSIG), or a range of about 0.1% to about 0.5% OSIG, or a range of about 0.4% to about 1% OSIG solution. For example, the concentration of IgG of about 0.01%, 0.05%, 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19%, or 20% OSIG solution. In one embodiment, a formulation was created using the OSIG Flebogamma 5% DIF to test its toxicity on human cells (FIG. 19). A concentration of 4 mg/ml of Flebogamma 5% DIF is nontoxic to the human epithelial cells and therefore is a suitable exemplary embodiment. The OSIG solutions may be fabricated with any commercially available IgG or pooled plasma IgG. Other embodiments formulated with nontoxic concentrations of OSIG solution using commercially available preparations are also suitable examples in accordance with the principles herein. In related aspects, the method further comprising the step of periodically delivering the treatment dosage to the treatment site for a treatment period defined by the response detected with the diagnostic kit at least one of twice per day and twice per month, or any other suitable time period to achieve an improvement in the immune or inflammatory condition or until the testing device indicates that the concentration is within normal ranges.

In an embodiment, a B-cell targeted eye composition can include a suitable B-cell composition, such as Rituximab, formulated as eye drops or other suitable delivery method to treat ocular or other mucosal diseases. For example, the B-cell targeted eye composition is immunomodulatory in the mucosa such as in the oral cavity, nasal cavity, bladder, tracheobronchial passages, ear canal and cavity, synovial (joint) cavity, vaginal cavity or over the skin.

In addition to applying the compositions described herein to the eye, these IgG compositions can be applied to the mucosa in other sites, for example in the oral cavity, nasal cavity, bladder, tracheobronchial passages, ear canal and cavity, synovial (joint) cavity, vaginal cavity and over the skin. The IgG compositions can be used to treat inflammatory and/or immune diseases at these mucosal sites. Specifically, the IgG compositions can be formulated for delivery to mucosa in various sites such as ocular, oral cavity, nasal cavity, bladder, tracheobronchial passages, ear canal and cavity, synovial (joint) cavity, vaginal cavity and over the skin to treat inflammatory and/or immune diseases at these mucosal sites

In yet another exemplary embodiment, B-cell targeted therapies (Rituximab, Ofatumumab, Belimumab, Atacicept, Tabalumab) can form a composition for treating immune eye disease, metabolic eye disease, allergic eye disease, traumatic eye disease, infectious eye disease and genetic eye disease, such as, but not limited to, ocular graft-versus-host disease (oGVHD), Steven Johnson syndrome, ocular cicatricial pemphigoid (OCP), mild, moderate and severe tear deficient dry eye disease (DED), meibomian gland disease, superior limbic keratoconjunctivitis (SLK), tear sufficient DED, floppy eyelid syndrome, neurotrophic eye disease, symptom-sign disconnect (discordant DED), neuropathic pain, thyroid eye disease (Grave's Ophthalmopathy), rheumatoid arthritis-related eye disease, lupus-related eye disease, Sjogren's syndrome, Secondary Sjogren's syndrome, ocular rosacea, allergic keratoconjunctivitis (vernal), viral keratoconjunctivitis (adenovirus EKC), Thygeson's keratitis, retinal gliosis, aniridia, keratitis or a postoperative/post-trauma ocular condition, peripheral ulcerative keratitis, keratitis, episcleritis, scleritis, Uveitis, retinitis, and glaucoma. For example, Rituximab can be formulated as eye drops or other suitable delivery method to treat ocular diseases.

### BRIEF DESCRIPTION OF THE DRAWINGS

FIG. 1 is boxplot graph that shows data on presence of ACPA in ocular surface washings of healthy subjects.

FIG. 2 is a boxplot graph that shows data on presence ACPA autoantibodies in ocular surface washings in patients with several ocular surface disease.

FIG. 3 is a graph that shows percent of all patients with a specific ocular surface disease who were positive for ACPA in ocular surface washings.

FIG. 4 is a boxplot graph that shows data on ACPA levels in tear fluid of Sjogren's syndrome patients before and after initiation of treatment.

FIG. 5 is a boxplot graph that shows data on ACPA levels in tear fluid of ocular GVHD patients before and after initiation of treatment.

FIG. 6 is a photograph of immunofluorescence staining of impression cytology specimen from the ocular surface showing the presence of citrullinated proteins in non-epithelial cells in Sjogren's syndrome patients. Sjogrens-cit/k14 or cit/NE→N=5; Healthy-cit/k14→N=2.

FIG. 7 is a photograph of immunofluorescence staining of impression cytology specimen from the ocular surface showing presence of citrullinated proteins in neutrophils in Sjogren's syndrome patients. cit/NE→N=2.

FIG. 8 is a photograph of immunofluorescence staining of impression cytology specimen from the ocular surface showing presence of citrullinated proteins in epithelial cells in ocular GVHD (oGVHD) patients. Def. oGVHD, cit/k14→N=4; None oGVHD, cit/k14→N=2.

FIG. 9 is a photograph of immunofluorescence staining of ocular surface mucoid films showing presence of inducible Fc-gamma receptors on neutrophils. Isolated neutrophils N=1; Pt mucoid N=2

FIG. 10 is a photograph of immunofluorescence staining of impression cytology specimen from the ocular surface showing presence of Fc-gamma receptors on non-epithelial cells (neutrophils and mononuclear cells). FcR+NE: N=5 different individuals, 4 different diseases.

FIG. 11 is a photograph of dot-blot-analysis and a graph showing that ACPA in tears is reactive to NET-citrullinated proteins.

FIG. 12 is a photograph of dot-blot-analysis and a graph showing that ACPA positive tears shows autoantibody polyclonality.

FIG. 13 is a photograph of immunofluorescence staining of ocular surface mucoid films showing presence of a citrullinated protein H4R3cit on patient's mucocellular aggregates.

FIG. 14 are photographs of mouse corneas showing ocular surface staining with fluorescein staining to show corneal epithelial disease. The data shows that ACPA antibodies can cause corneal epithelial disease, however not all ACPA antibodies can do so.

FIG. 15 is a photograph of immunofluorescence staining of impression cytology specimen from the ocular surface of mouse cornea showing ACPA antibody #1 causes NETosis and induces citrullination on mouse cornea epithelium.

FIG. 16 are photographs of mouse corneas showing ocular surface staining with fluorescein staining to show corneal epithelial disease. The data shows that ACPA-induced pathological effect on cornea is mediated via Fc receptors. Mouse IgG competes with ACPA antibodies for binding to Fc receptors, and Fc receptor blocker blocks all Fc receptors. This competitive and peptide blocking may have abrogated pathological effect of ACPA on the cornea, suggesting that using methodologies that prevent the interaction of ACPA antibodies with Fc receptors, and may be a potential therapeutic strategy in treating citrullination related eye diseases.

FIG. 17 is a photograph of immunofluorescence staining of neutrophils showing that ACPA-H4R3cit induces NETosis in vitro.

FIG. 18 is data showing cytotoxicity of OSIG on human corneal epithelial cells. OSIG concentrations from 1 mg/ml to 4 mg/ml do not cause cytotoxicity as evidenced by no increase in LDH levels.

FIG. 19 provides an exemplary image of an eye with high autoantibodies (ACPA) in tear fluid and severe ocular surface disease.

FIGS. 20A and 20B provide exemplary images of an eye with high autoantibodies (ACPA) in tear fluid but no systemic autoimmune disease and serum negative for ACPA autoantibodies and Rheumatoid arthritis.

FIGS. 21A and 21B provide exemplary images of an eye with asymmetric eye disease; eye with more severe ocular surface disease has higher autoantibody (ACPA) levels in tear fluid.

FIGS. 22A and 22B provide images from a 56 year old female with severe tear deficiency and severe ocular surface disease due to ocular Graft-VS-Host Disease (case study 1). These images demonstrate that OSIG treatment reduced the signs and symptoms of severe dry eye as well as reduced the level of an inflammatory biomarker in tear fluid.

FIG. 23 provides images from a 31 year old female with severe tear deficiency and severe ocular surface disease due to ocular Graft-VS-Host Disease (case number 2). These images demonstrate that OSIG treatment reduced the symptoms of neurotrophic keratinitis.

FIG. 24 provides images from a 74 year old male with neurotropic keratitis due to history of prior LASIK eye surgery in the left eye (case study 3). These images demonstrate that OSIG treatment reduced the signs and symptoms of severe dry eye as well as reduced the level of an inflammatory biomarker in tear fluid.

FIG. 25 provide images from a 29 year old male with tear deficiency and severe ocular surface disease due to Steven Johnsons Syndrome (case study 6). These images demonstrate that OSIG treatment reduced the signs and symptoms of severe dry eye after Steven Johnson syndrome . . .

### DETAILED DESCRIPTION

In accordance with the principles herein, autoantibodies such as anti-citrullinated protein antibodies (ACPAs) were found to be present in tear fluid of patients with eye diseases such as dry eye disease (DED), sjogren's syndrome, meibomian gland disease, superior limbic keratoconjunctivitis (SLK), ocular cicatricial pemphigoid, Steven Johnson's syndrome, graft-versus-host disease, peripheral ulcerative keratitis, episcleritis, scleritis and such autoimmune diseases. Surprisingly, the ACPA autoantibodies were present in the tear fluid of patients even though ACPA and rheumatoid factor were not detected in serum, suggesting that the tear fluid autoantibodies are produced locally in/around the eye tissues. This result was surprising because one would not have expected ACPA autoantibodies to be present in tear fluid of eye disease patients as their serum did not have ACPA or rheumatoid factor and they did not have evidence of systemic autoimmune disease. Patients with unilateral disease had higher level of ACPA in the tear fluid of eye that had more severe disease. Further, eyes with very high ACPA levels in the tear fluid frequently had evidence of severe disease such as corneal melting and scars and were inadequately controlled despite aggressive treatments. In the studies described herein, citrullinated proteins over the ocular surface of these patients were identified, and also the presence of Fc receptors. Laboratory experiments confirmed that exposure of mouse corneas to ACPAs can cause ocular surface disease, and that the deleterious effects of ACPAs on ocular surface disease were reduced by targeting their interaction with eye tissues by using IgG or Fc receptor block. Taken together, pooled IgG and/or synthetic variants thereof are a suitable form of a therapy for eye diseases caused by or affected by autoantibodies. In addition, polyclonality of autoantibodies was identified in the tear fluid (e.g presence of cit histone, cit alpha-enolase, cit fibrinogen etc in tear fluid, which makes pooled IgG an attractive therapy because targeting individual antibodies unattractive and laborious.

As used in this specification and the appended claims, the singular forms “a,” “an” and “the” include plural references unless the content clearly dictates otherwise.

In an exemplary embodiment, an ophthalmic formulation comprises: (a) a pharmaceutically active compound that is capable of reducing the amount of autoantibodies over the ocular surface or inside the eye, such autoantibodies being generated in response to citrullinated-protein (ACPA autoantibodies due to citrullination of protein) or autoantibodies being generated in responses to homocitrullinted-protein (anti-CarP antibodies due to carbamylation of protein) or autoantibodies being generated in response to to native proteins; (b) optionally a second pharmaceutically active compound selected from the group consisting of a PAD enzyme inhibitor, NETs dismantling agent and Fc receptor blockers, and a combination thereof; and (c) a pharmaceutically acceptable ophthalmic excipient. Unless the context requires otherwise, the term “pharmaceutically active compound” refers to a compound that is pharmaceutically active when applied topically to ocular surface or injected inside the eye. Thus, for example, the term a pharmaceutically active NSAID refers to an NSAID that is pharmaceutically active when applied topically to ocular surface or injected inside the eye.

Still another aspect of the disclosure provides an ophthalmic formulation comprising of: (a) a pharmaceutically acceptable ophthalmic excipient; (b) a therapeutically effective amount of a pharmaceutically active compound comprising immunoglobulin G (IgG); and (c) optionally a second pharmaceutically active compound selected from the group consisting of a steroid, an anti-inflammatory agent, a mucolytic agent, a PAD enzyme inhibitor, NETs dismantling agent and a Fc receptor blocker, and a combination thereof and a combination thereof. The pharmaceutically active compound comprising immunoglobulin G (IgG) is capable of treating a clinical condition selected from the group consisting of inflammatory and immunological ocular surface disease that can cause symptoms of ocular discomfort, a dry eye syndrome, keratitis, symblepheron formation, fornix foreshortening, eyelid margin/conjunctival keratinization, and subconjunctival fibrosis. “A therapeutically effective amount” means the amount of a compound that, when administered to a mammal for treating a disease or a clinical condition, is sufficient to effect such treatment for the disease. The “therapeutically effective amount” will vary depending on the compound, the disease or the clinical condition and its severity and the age, weight, etc., of the mammal to be treated. “Treating” or “treatment” of a clinical condition or disease includes: (1) preventing the clinical condition or disease, i.e., causing the clinical symptoms of the condition or disease not to develop in a mammal that may susceptible to the clinical condition or disease but does not yet experience or display symptoms of the clinical condition or disease; (2) inhibiting the clinical condition or disease, i.e., arresting or reducing the development of the clinical condition or disease or its symptoms; or (3) relieving the clinical condition or disease, i.e., causing regression of the clinical condition or disease or its symptoms.

Still another aspect of the disclosure provides an ophthalmic formulation comprising: (a) a pharmaceutically acceptable ophthalmic excipient; (b) a therapeutically effective amount of a pharmaceutically active compound comprising pooled human immunoglobulin G (IgG); and/or (c) a therapeutically effective amount of pooled human plasma proteins (in dilution ranging from 0.1% to 99% of human plasma protein) and pooled human plasma lipids (in dilution ranging from 0.1% to 99%).

Ophthalmic formulations of the disclosure are useful for treatment of various clinical condition associated with inflammatory and immunological ocular surface diseases. Exemplary clinical conditions that can be treated by ophthalmic formulations of the disclosure include, but are not limited to, ocular surface disease that can cause symptoms of ocular discomfort, a dry eye syndrome, mucocellular aggregates/debris in tear film, symblepheron formation, fornix foreshortening, eyelid margin/conjunctival keratinization, and subconjunctival fibrosis. More specific types of ocular surface diseases include ocular graft-versus-host disease (oGVHD), Steven Johnson syndrome, ocular cicatricial pemphigoid (OCP), mild, moderate and severe tear deficient dry eye disease (DED), meibomian gland disease, superior limbic keratoconjunctivitis (SLK), tear sufficient DED, floppy eyelid syndrome, neurotrophic eye disease, symptom-sign disconnect (discordant DED), neuropathic pain, thyroid eye disease (Grave's Ophthalmopathy), rheumatoid arthritis-related eye disease, lupus-related eye disease, Sjogren's syndrome, Secondary Sjogren's syndrome, ocular rosacea, allergic keratoconjunctivitis (vernal), aniridia, keratitis due to sterile inflammation or due to a viral, bacterial or fungal infection, a postoperative/post-trauma ocular condition, peripheral ulcerative keratitis, episcleritis, scleritis, Uveitis and glaucoma. Exemplary postoperative/post-trauma ocular conditions that can be treated using ophthalmic formulations of the disclosure include, but are not limited to, an ocular condition associated with post-ocular surface reconstruction surgery, antimetabolite application to eye surface, pterygium surgery, glaucoma surgery, cataract surgery, laser vision correction surgery (LASIK, LASEK, EPI-LASIK) keratoprosthesis surgery and radiation injury.

Other ocular clinical conditions that can be treated using ophthalmic formulations of the disclosure include, but are not limited to, dry eye syndrome (keratoconjunctivitis sicca), sjogren's syndrome, congenital alacrima, xerophthalmia (dry eye from vitamin A deficiency), keratomalacia, thyroid eye disease, ocular rosacea, eyelid disorders, meibomian gland disease, meibomian gland dysfunction, ectropion, blepharitis, blepharochalasis, sarcoidosis, stye, hordeolum, chalazion, ptosis, pterygium, eyelid edema, eyelid dermatitis, trichiasis, madarosis, dacryoadenitis, stevens-johnson syndrome, ocular graft versus host disease, dacryocystitis, conjunctivitis, keratoconjunctivitis, blepharoconjunctivitis, blepharokeratoconjunctivitis, allergic conjunctivitis, vernal conjunctivitis, conjunctival suffusion, conjunctivochalasis, subconjunctival hemorrhage, pterygium, pinguecula, chemosis, iritis, iridocyclitis, anterior uveitis, glaucoma, red eye, keratitis, scleritis, episcleritis, peripheral ulcerative keratitis, neurotrophic keratitis, neurotrophic eye disease, corneal ulcer, ulcerative keratitis, corneal abrasion, photokeratitis, ultraviolet keratitis, exposure keratitis, superficial punctuate keratitis, thygeson's superficial punctuate keratopathy, herpes simplex keratitis, herpes zoster keratitis, acne rosacea, post-operative inflammation following ocular surgery (i.e. eyelid surgery, cataract surgery, corneal surgery, refractive surgery including photorefractive keratectomy, glaucoma surgery, lacrimal gland surgery, conjunctival surgery, eye muscle surgery), ocular surface conditions caused by chemical burns, thermal burns or physical trauma, ocular conditions caused by the following autoimmune or vascular disorders: rheumatoid arthritis, juvenile rheumatoid arthritis, ankylosing spondylitis, reiter's syndrome, enteropathic arthritis, psoriatic arthritis, discoid and systemic lupus erythematosus, multiple sclerosis, graves' disease, antiphospholipid syndrome, sarcoidosis, wegner's granulomatosis, behcet's syndrome, polyarteritis nodosa, takayasu's arteritis, dermatomyositis, psoriasis, relapsing polychondritis, vasculitis and sickle cell-anemia.

In some embodiments, ophthalmic formulations of the disclosure are used to treat a dry eye syndrome. There are two major classes of dry eye syndrome: (i) aqueous tear-deficient dry eye (ADDE) and (ii) evaporative dry eye (EDE). There are also cases of mixed mechanism dry eye (i.e., both ADDE and EDE). ADDE is primarily due to failure of lacrimal tear secretion. ADDE can be further subdivided into Sjogren syndrome dry eye (where the lacrimal and salivary glands are targeted by an autoimmune process, e.g., rheumatoid arthritis) and non-Sjogren's syndrome dry eye (lacrimal dysfunction, but the systemic autoimmune features of Sjogren's syndrome are excluded, e.g., age-related dry eye). In contrast, EDE is primarily due to excessive water loss from the exposed ocular surface in the presence of normal lacrimal secretory function. Its causes can be extrinsic (e.g., ocular surface disorder due to some extrinsic exposure, contact lens wear or vitamin A deficiency) or intrinsic (e.g., Meibomian gland dysfunction and disorders of eyelid aperture). Meibomian glands secrete a mixture of lipids and other components that form the outer layer of the preocular tear film. This lipid layer functions to decrease tear film evaporation. Meibomian gland dysfunction (MGD) leads to evaporative dry eye disease. One of the most well recognized clinic finding in MGD is the presence of numerous telangiectatic blood vessels coursing across the eyelid margin. MGD can also accompany tear deficient dry eye disease, as seen in ocular graft-versus-host-disease (oGVHD). Other specific dry eye syndromes that can be treated using compositions of the disclosure include keratoconjunctivitis, dry eye caused by conjunctivitis, dry eye caused by allergic conjunctivitis, dry eye caused by blepharitis, dry eye caused by keratitis, dry eye caused by dacryoadenitis, dry eye caused by ocular rosacea, dry eye caused by boehm syndrome, dry eye caused by conjunctivochalasis, dry eye caused by blepharoconjunctivitis, dry eye caused by blepharokeratoconjunctivitis, dry eye caused by superficial punctuate keratitis, dry eye caused by thygeson's superficial punctuate keratopathy, dry eye caused by oGVHD, Sjogren's dry eye syndrome, dry eye caused by Stevens-Johnson syndrome, MGD, dry eye caused by meibomian gland disease, vitamin A deficiency induced dry eye, pharmacological induced dry eye (i.e. hormone replacement therapy, blood pressure medication, antihistamine, antidepressants, anticholinergic medications, glaucoma medication, antihypertensives, diuretics, sedatives, isotretinoin, nasal decongestants, oral contraceptives, beta-blockers, phenothiazines, atropine, pain relieving opiates), pregnancy induced dry eye, LASIK surgery or refractive surgery induced dry eye, dry eye induced by collagen vascular diseases (i.e. systemic lupus erythematosus, Wegener's granulomatosis, rheumatoid arthritis, relapsing polychondritis), dry eye caused by the infiltration of the lacrimal glands by tumors or sarcoidosis, dry eye caused by postradiation fibrosis of tear producing glands, dry eye caused by lacrimal gland, meibomian gland, or goblet cell ablation, dry eye caused by sensory denervation, dry eye caused by thermal or chemical burns, dry eye caused by underlying diabetic conditions, dry eye caused by viral, fungal, or bacterial infection, dry eye caused by prolonged contact lens use, dry eye caused by eyelid disorders or injury to the eyelid (i.e. bulging eyes, drooping eyelid), dry eye caused by corneal dystrophy, dry eye caused by autoimmune disorders, age-induced dry eye, and a combination thereof.

In some particular embodiments, ophthalmic formulations of the disclosure are used to treat Meibomian gland dysfunction (MGD). Yet in other embodiments, ophthalmic formulations of the disclosure are used to treat aqueous tear-deficient dry eye (ADDE). In some instances, methods for treating ADDE comprise treating a patient in need of a treatment for Sjogren dry eye syndrome, ocular Graft-Versus-Host-Disease (oGVHD) or non-Sjogren dry eye syndrome. Yet in other embodiments, methods for treating dry eye syndrome comprise treating a patient in need of a treatment of evaporative dry eye (EDE). Still in other embodiments, methods of the disclosure include treating a patient in need of a treatment for mixed mechanism dry eye consisting of ADDE and EDE. Yet still in other embodiments, methods of the disclosure include treating a patient suffering from dry eye syndrome due to a complication of refractive eye surgery or is attributable to one or more of the following causes: vitamin A deficiency, ocular surface disorders, allergy, aging, contact lens usage, medication usage or eyelid disorders.

Two forms of post-translational modification (PTM), citrullination and carbamylation, result in the generation of two non-standard amino acids in polypeptides, citrulline and homocitrulline, respectively, which are chemically highly related. There is increasing evidence for a pathophysiological role of protein citrullination and carbamylation, such as a role for autoantibodies to these proteins in Rheumatoid arthritis. However, the role of citrullination and carbamylation and ACPA in the context of ocular surface disease has not been described previously. The studies provided herein identify the presence of ACPA in ocular surface fluid.

In some embodiments, the ophthalmic formulation comprises immunoglobulin G (IgG) as a pharmaceutically active ingredient. Type of IgG that can be used in ophthalmic formulations of the disclosure include, but are not limited to, (i) serum/plasma-derived pooled immunoglobulin G (OSIG); (ii) autologous IgG purified from autologous plasma/serum; (iii) multimerized IgG1 Fc molecule (IgG1 Fc hexamer); (iv) IgG2a Fc multimers (stradomers); (v) Multivalent Fc structures; (vi) glycoengineered sialylated IgG; and (vii) IgG-Fc Glycosylation, or a combination thereof. In some embodiments, the commercially available OSIG preparations are formulated with ophthalmic excipients (diluted or concentrated, as needed) to achieve a IgG concentration that is three to six times the concentration of IgG that is normally present (either physiologically or pathologically) at the site of application, and then dispensed as eye drops, injectables or other appropriate delivery method. Examples of commercially available IgG solutions include “Bivigam 10%, Cuvitru (20%), Flebogamma DIF 5% & 10%, Gammagard Liquid 10%, Gammagard S/D 5% & 10%, Gammaked 10%, Gammaplex 5% & 10%, Gamunex—C 10%, Hizentra 20%, HYQVIA 10%, Octagam 5% & 10%, Privigen 10%”. These commercially available IgG solutions may be compounded with an ophthalmic excipient to the desired IgG concentration, e.g. 4 mg/ml, and the desired pH of at least 6.0 to fabricate the disclosed OSIG compositions.

In some embodiments, the ophthalmic formulation consists of functional equivalents of whole IgG antibody, such as antigen binding fragments in which one or more antibody domains are truncated or absent (e.g., Fv, Fab, Fab′, or F (ab) 2 fragments), as well as genetically-engineered antibodies or protein binding fragments thereof, including single chain antibodies or antibodies that can bind to more than one epitope (e.g., bi-specific antibodies), or antibodies that can bind to one or more different antigens (e.g., bi- or multi-specific antibodies), can also be employed in the disclosure.

Immunoglobulins are a group of closely related glycoproteins composed of 82%-96% protein and 4%-18% carbohydrate. These glycoproteins of about 150 kDa are present in plasma at a mean concentration of 7 to 12 g/L depending upon individual variations and level of environmental exposure to antigens. The immunoglobulin G (IgG), a major effector molecule of the humoral immune response in man accounts for about 75% of the total Igs in the plasma of healthy individuals. The basic Ig molecule has a four-chain structure, comprising two identical heavy (H) chains and two identical light (L) chains, linked together by inter-chain disulfide bonds. IgG possesses dual functions characterized by the capacity to recognize and react specifically with the antigen and to perform a series of non-specific effector functions in which the antigen is rendered harmless and eventually eliminated. This functional dichotomy of IgG is reflected in the structure of the molecule that comprises two variable regions responsible for antigen binding (Fab), and a constant region (the Fc or crystallisable fragment) that mediates the specific effector functions. The presence of a complex oligosaccharide structure modulates the functions of IgG, especially the activation of complement and binding to FcγR. In some embodiments, the ophthalmic formulation of the disclosure will consist of immunoglobulin G (IgG) with enhanced sialylation (Sialylated IgG), such sialylation being achieved using standard chemical processes.

In some embodiments, the concentration of IgG in the ophthalmic formulation of disclosure is defined by a 10% ocular surface immunoglobulin (OSIG) solution, or any other suitable concentration. OSIGs are a therapeutic preparation of pooled normal polyspecific human IgGs obtained from serum/plasma of large numbers of healthy donors. The preparation contains antibodies to microbial antigens, self-antigens (natural autoantibodies) and anti-idiotypic antibodies which recognize other antibodies. Plasma used in the production of OSIG comes from two origins: approximately 20 percent is from blood donors, and the other 80 percent is from plasma donors. Individual plasmas are pooled; the pool size is a minimum of 1000 donors, but may be up to 100,000 donors. The many thousands of donors who contribute to a typical pool of plasma used for isolation of immunoglobulin represent a wide range of antibody specificities against infectious agents such as bacterial, viral and also a large number of self-antigens reflecting the cumulative exposure of the donor population to the environment. Preparations of OSIG consist of intact IgG molecules with a distribution of IgG subclasses (IgG1, IgG2, IgG3 and IgG4) corresponding to that of normal serum. The primary purification processes for OSIG production may include: (i) Fractionation e.g. Polyethylene glycol (PEG) is a synthetic polymer that is also used to separate proteins by fractional precipitation from a natural mixture like plasma by an exclusion mechanism; and (ii) Chromatography e.g. anion exchange chromatography, hydrophobic Charge Induction Chromatography or Size exclusion chromatography. The biological effects of OSIG that may contribute to the efficacy of the ophthalmic formulation of disclosure include: (i) Functional Blockade of Fc Receptors. The saturation of Fc receptors by OSIG leads to decreased cellular destruction as a consequence of Fc-mediated biological effects, (ii) Autoantibody Neutralization and Inhibition of Autoantibody Production. OSIG preparations contain anti-idiotypic antibodies, i.e., antibodies that are able to interact specifically with the variable region (antigen recognition site) of autoantibodies. This interaction has the potential to neutralize an autoantibody and to hamper its production via binding to autoreactive B lymphocytes, (iii) Complement Inhibition. The Fc portion of OSIG can bind the C3b and C4b fragments of complement, and thereby inhibit their tissue deposition as well as the generation of the C5 convertase, (iv) Modulation of Cytokine (including IL-1, -2, -3, -4, -5, -10, TNF-alpha, and GM-CSF) and Cytokine Antagonist Production (IL-1 receptor antagonist); and (v) Signaling through the Inhibitory Fc Receptor, Fc gamma RIIB.

The amount of IgG in the ophthalmic formulation of the disclosure can range from about 0.01 mg/mL to about 1 g/mL, typically from about 1 mg/mL to about 10 mg/mL. In certain embodiments the concentration or amount of IgG may be about 0.01 mg/mL, in other embodiments the concentration or amount of IgG may be about 0.05 mg/mL. In certain embodiments the concentration or amount of IgG may be about 1 mg/mL, in other embodiments the concentration or amount of IgG may be about 10 mg/mL. In various embodiments, the concentration or amount of IgG may be 1.0 mg/mL. 1.5 mg/mL, 2.0 mg/mL, 2.5 mg/ml, 3.0 mg/ml, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, or 10 mg/mL,

The present disclosure covers the presence of autoantibodies on the ocular surface (tear fluid) or intraocular (aqueous and vitreous) in various eye diseases of which Anti Citrullinated Protein Antibodies (ACPAs) are the prototype example. Autoantibodies that may be present and are emcompassed by this disclosure include antibodies to the following proteins: β2-Glycoprotein, C1q, CENP-B (Centromere Protein B), CENP-A (Centromere Protein A), Jo-1, Ku, Mi-2, Myeloperoxidase (MPO), PCNA (Proliferating Cell Nuclear Antigen A), PL-12 (Alanyl-tRNA synthetase), PM/Scl 100, Proteinase 3, RNP (Ribonucleoprotein), RNP/Smith (RNP/Sm), Ribosomal P, Scl-70, Sm, SSB/La (Sjögren's Syndrome-related antigen B/La), SSA/Ro60 (Sjögren's Syndrome-related antigen A/Ro60 kDa), SSA/Ro52 (Sjögren's Syndrome-related antigen A/Ro52 kDa). Although not designed to be exhaustive/complete, autoantibodies (ACPA or non-ACPA) covered under this disclosure may be derived from the proteins or their fragments (citrullinated or native) listed in Table 1:

TABLE 1

| | | | |
|---|---|---|---|
| Aggrecan | ssRNA | La/SSB | Ro/SSA(60 KDa) |
| alpha Fodrin(Sptan1) | dsDNA | Laminin | S100 |
| alpha-actinin | EBNA1 | LC1 | Scl-70 |
| Amyloid | Elastin | LKM1 | Sm |
| AQP4 recombinant | Entaktin EDTA | M2 antigen | Sm/RNP |
| BP1 | Factor I | Matrigel | SmD |
| C1q | Factor P | MDA5 | SmD1 |
| Cardolipin | Factor B | Mi-2 | SmD2 |
| CENP-A | Factor D | Mitochondrial antigen | SmD3 |
| CENP-B | Factor H | MPO | SP100 |
| Chondroitin Sulfate C | Fibrinogen IV | Muscarinic receptor | Sphingomyelin |
| Chromatin | Fibrinogen S | Myelin basic protein (MBP) | SPR54 |
| Collagen I | Fibronectin | Myelin-associated | ssDNA |
| | | glycoprotein-FC | |
| Collagen II | GBM (disso) | Myosin | T1F1 GAMMACollagen |
| Collagen III | Genomic DNA | Nucleolin | Thyroglobulin |
| Collagen IV | Gliadin (IgG) | Nucleosome antigen | TNFa |
| Collagen V | Glycated Albumin | Nup62 | Topoisomerase I |
| Collagen VI | GP2 | PCNA | TPO |
| complement C1q | gP210 | Peroxiredoxin 1 | TTG |
| complement C3 | Histone H1 | Phophatidylinositol | U1-snRNP-68 |
| complement C3a | Histone H2A | PL-12 | U1-snRNP-A |
| complement C3b | Histone H2B | PL-7 | U1-snRNP-BB′ |
| complement C4 | Histone H3 | PM/Scl-100 | U1-snRNP-C |
| complement C5 | Histone H4 | PM/Scl-75 | Vimentin |
| complement C6 | Hemocyanin | POLB | Vitronectin |
| complement C7 | Heparan HSPG | PR3 | β2-glycoprotein I |
| complement C8 | Heparin | Proteoglycan | β2-microglobulin |
| complement C9 | Heperan Sulfate | Prothrombin protien | IgA - human and |
| | | | mouse |
| CPR antigen(human) | Histone (total) | Ribo phasphoprotein P1 | IgE- human |
| Cytochrome C | Intrinsic Factor | Ribo phasphoprotein P2 | IgG - human and |
| | | | mouse |
| Decorin-bovine | Jo-1 | Ribo phasphoprotein P0 | IgM - human and |
| | | | mouse |
| DGPS | KU (P70/P80) | Ro/SSA (52 KDa) | Anti-IgG, IgA and |
| | | | anti-IgM |

“Pharmaceutically acceptable excipient” or “pharmaceutically acceptable ophthalmic excipient” means an excipient that is useful in preparing a pharmaceutical composition of the disclosure. Such an excipient is considered by one skilled in the art as being generally safe, non-toxic and neither biologically active nor otherwise undesirable, and includes excipient that is acceptable for veterinary use as well as human pharmaceutical use. A “pharmaceutically acceptable excipient” as used in the specification and claims includes both one and more than one such excipients. Exemplary pharmaceutically acceptable excipients include a salt (such as sodium chloride) or a tonicity agent, gum, resin, a solvent such as water, a non-aqueous solvents (such as an alcohol, an oil, a buffer solution to maintain pH, a pH modifying agent (e.g., a base such as sodium hydroxide, and an acid such as hydrochloric acid), an emulsifier, a thickening agent, a micro or a nano-emulsion forming agent, a preservative, a surfactant, etc. Exemplary pharmaceutically acceptable excipients that can be used in ophthalmic formulations of the disclosure include, but are not limited to, water, benzyl alcohol, sodium hydroxide, hydrochloric acid, Castrol oil, citrate buffer, Tris buffer, phosphate buffer, as well as other excipients known to one skilled in the art.

In some embodiments, ophthalmic formulations of the disclosure can also include salts such as sodium chloride. Yet in other embodiments, the ophthalmic formulation of the disclosure can also include a non-aqueous solvent such as benzyl alcohol, ethanol, or other non-aqueous solvents known to one skilled in the art.

Still in other embodiments, pH of the ophthalmic formulations of the disclosure is adjusted from pH of about 5.0 to pH of about 8.5, typically from pH of about 5.0 to pH of about 8.0, and often from pH of about 5.0 to pH of about 7.5. Additional, exemplary pH rangers are about pH 6 to about pH 8 or about pH 6.2 to about pH 7.2, about pH 6.4 to about pH 7.4, or about pH 6.5 to about pH 7.5, about pH 6.6 to about pH 7.6, about pH 6.8 to about pH 7.8. For example, the pH is about 5.0. is about 5.1, or about 5.2, or about 5.3, or about 5.4, or about 5.5, or about 5.6, or about 5.7, or about 5.8, or about 5.9 about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 7.6, or about 7.7, or about 7.8, or about 7.9, or about 8.0. The pH of ophthalmic formulations of the disclosure can be adjusted using, for example, sodium hydroxide and/or hydrochloric acid as needed to achieve a desired pH level.

The ophthalmic formulation can be formulated as an eye drop, topical liquid, an ointment, emulsion, suspension or a gel (e.g., IgG sodium in hydrogel). The ophthalmic formulation can also be formulated as a nano-emulsion of oil or a suspension. In addition, the ophthalmic formulation can be formulated as an injectable formulation.

In some embodiments, ophthalmic formulations of the disclosure are preservative free and are formulated for a single-use or in a multi-dose vials. If a preservative is used, suitable preservatives include, but are not limited to, benzalkonium, purite, chlorobutanol, sodium perborate, stabilized oxychloro complex (SOC), Polyquaternium-1 (Polyquad, PQ-1), Thimerosal, Benzyl alcohol, Sorbic acid, Methyl/propyl paraben, Chlorhexidine, Disodium EDTA, sofZia, and other preservatives known to one skilled in the art of ophthalmology or ophthalmic formulation chemistry.

When present, the second pharmaceutically active compound is selected from the group consisting of a steroid, an anti-inflammatory agent, a mucolytic agent and a combination thereof. Exemplary steroids that are suitable in ophthalmic formulations of the disclosure include, but are not limited to, methylprednisone, prednisone, dexamethasone, loteprednol etabonate, fluocinolone, difluprednate, fluorometholone, medrysone, fluocinolone, rimexolone triamcinolone, and a combination thereof. When a steroid is used in ophthalmic formulations of the disclosure, the amount of steroid present in the formulation ranges from about 0.01% w/w to 2% w/w; typically from about 0.05% w/w to 1% w/w, and often from about 0.1% w/w to about 0.3% w/w. It should be appreciated that the scope of the disclosure is not limited to these particular ranges of the amount of steroid. In particular, the amount of steroid present in ophthalmic formulations of the disclosure generally depends on the steroid used. For example, the amount of steroid present can vary depending on the activity of the particular steroid used, molecular weight of the steroid, as well as the purpose of using a steroid in ophthalmic formulations of the disclosure.

Exemplary anti-inflammatory agents that are suitable in ophthalmic formulations of the disclosure include, but are not limited to, cyclosporine, Lifitegrast®, tacrolimus, interleukin 1 receptor antagonist (anakinra), other NSAIDs such as ketorolac, diclofenac, flurbiprofen, bromfenac, nepafenac, and a combination thereof. When an anti-inflammatory agent is used in ophthalmic formulations of the disclosure, the amount of anti-inflammatory agent present in the formulation ranges from about 0.01% w/w to 2% w/w; typically from about 0.05% w/w to 1% w/w, and often from about 0.1% w/w to about 0.3% w/w. It should be appreciated that the scope of the disclosure is not limited to these particular ranges of the amount of anti-inflammatory agent. In particular, the amount of anti-inflammatory agent present in ophthalmic formulations of the disclosure generally depends on the particular anti-inflammatory agent used, such as the activity of the particular anti-inflammatory agent used, molecular weight of the anti-inflammatory agent, etc.

Exemplary mucolytic agents that are useful in ophthalmic formulations of the disclosure include, but are not limited to, N-acetylcysteine, Nacystelyn, Dextran, DNaseI (dornase alpha), gelsolin, thymosinβ4, 14- and 15-member macrolide antibiotics (e.g., erythromycin, a non-antimicrobial derivative of erythromycin (e.g., EM703 and EM900), clarithromycin, roxithromycin, Fidaxomicin, Telithromycin and azithromycin), and a combination thereof. It should be appreciated that when an antibiotic is used, it is primarily used for its mucolytic activity. When a mucolytic agent is used in ophthalmic formulations of the disclosure, the amount of mucolytic agent present in the formulation ranges from about 0.01% w/w to 2% w/w; typically from about 0.05% w/w to 1% w/w, and often from about 0.1% w/w to about 0.3% w/w. It should be appreciated that the scope of the disclosure is not limited to these particular ranges of the amount of mucolytic agent. In particular, the amount of mucolytic agent present in ophthalmic formulations of the disclosure generally depends on the particular mucolytic agent used, such as the activity of the mucolytic agent, molecular weight of the mucolytic agent, etc.

Ophthalmic formulations of the disclosure can be homogeneous or heterogeneous. In some embodiments, ophthalmic formulations of the disclosure contain an oil or a fatty acid ester. A fatty acid ester has the meaning commonly understood in the art, being an ester formed between an alcohol and a fatty acid. Exemplary fatty acid esters that are useful in formulations of the disclosure include, but are not limited to, triglyceride esters commonly known as vegetable oils, mono and diglyceride esters of fatty acids, fatty acid methyl esters, as well as other fatty acid esters that are known to one skilled in the art. It should be appreciated the fatty acid ester can be a mixture of several chemical compounds or an essentially pure compound. Typically, the fatty acid ester is a vegetable oil. Particular examples of vegetable oils that can be used include, but are not limited to, castor oil, sesame oil, soybean oil, cottonseed oil, olive oil, peanut oil, safflower oil, sunflower oil, palm oil, palm kernel oil, canola oil, and Miglyol Oil®.

Various vehicles can be used in the ophthalmic formulations of the disclosure. These vehicles include, but are not limited to, purified water (water), polyvinyl alcohol, povidone, hydroxypropyl methyl cellulose, poloxamers, carboxymethyl cellulose, hydroxyethyl cellulose, polyols, sodium hyaluronate, pluronics, corbopol, cyclodextrin and a mixture of two or more thereof. The vehicle is used in the formulation in amounts as needed to provide the concentration of the active compound(s) disclosed herein. In one particular embodiment, the vehicle comprises water.

In some embodiments of this disclosure, an emulsion stabilizing polymer is used. While not intending to limit the scope of the disclosure, emulsion stabilizing polymers generally contain hydrophilic groups such as cellulose, sugars, ethylene oxide, hydroxide, carboxylic acids or other polyelectrolytes. Without being bound by any theory, it is believed that these polymers help to stabilize emulsions by increasing the viscosity of the formulation as well as by reducing the interfacial tension. Surfactants such as polysorbate 80 or other surfactants acceptable for Opthalmics can be used to stabilize emulsions, Some examples of emulsion stabilizing polymers useful in this disclosure include, but are not limited to, carbomers, Pemulen®, sodium carboxymethylcellulose, hydroxypropylmethylcellulose, povidone, polyvinyl alcohol, polyethylene glycol and a mixture of two or more thereof.

The ophthalmic formulations of the present disclosure can be packaged in various package forms known in the field of topical ophthalmic. In one particular embodiment, the ophthalmic formulation is packaged in sterile, preservative-free single-use packs or vials or containers (i.e., the unit dose vials). Each vial, for example as small as a 0.9 mL, may be made of low density polyethylene so as to contain a small quantity of the formulation, e.g., 0.4 mL for a single use. This way, where the ophthalmic formulation is sterilized and contained in disposable single-dose containers for topical use in drop form, multiple vials in the form of a set of 30 vials, 60 vials and so on can be packaged in a tray with a lid, for example, a polypropylene tray with an aluminum peelable lid. The entire contents of each tray can be dispensed intact, and one vial or pack is used each time and immediately discarded after each use. For example, plastic ampules or vials or containers can be manufactured using blow-fill-seal (BFS) technology. The BFS processes may involve plastic extrusion, molding, aseptic filling, and hermetic sealing in one sequential operation and those processes are known in the art. In another embodiment, the formulation is packaged in multi-dose vials such that the materials can be dispensed as sterile at each time using specialized container/closure maintaining the sterility integrity. In yet another embodiment, the ophthalmic formulation is packaged in conventional vials/containers as a sterile product.

In some embodiments, the dosage form of the disclosure is eye drops of heterogeneous aqueous solution, eye drop formulations.

In one particular embodiment, ophthalmic formulation comprising IgG (OSIG) is formulated as eye drops and are used to treat inflammatory and immunological ocular surface disease that can cause symptoms of ocular discomfort, mucocellular aggregates/debris in tear film, symblepheron formation, fornix foreshortening, eyelid margin/conjunctival keratinization, or subconjunctival fibrosis. Specific clinical conditions that can be treated using ophthalmic formulations of the disclosure include such diseases as ocular graft-versus-host disease (oGVHD), Steven Johnson syndrome, ocular cicatricial pemphigoid (OCP), mild, moderate and severe tear deficient dry eye disease (DED) (secondary to sjogrens syndrome, non-sjogrens syndrome, idiopathic and other causes), meibomian gland disease (dysfunction or atrophy), superior limbic keratoconjunctivitis (SLK), tear sufficient DED (concordant or discordant), floppy eyelid syndrome, neurotrophic eye disease, aniridia and postoperative/post-trauma pathologies, e.g., after ocular surface reconstruction surgery, antimetabolite application to eye surface (mitomycin-C, 5-fluorouracil and others) or keratoprosthesis surgery or radiation injury.

In yet another aspect of the disclosure provides a method for treating an ocular disease to a subject in need of such a treatment. Such a method generally involves administering a therapeutically effective amount of a sub-anticoagulant amount of IgG ophthalmic formulation to the subject to treat an ocular disease. In some embodiments, the IgG ophthalmic formulation is administered at least twice a day to said subject. Still in other embodiments, the IgG ophthalmic formulation is administered at least twice a month to said subject. The ophthalmic formulation can be an aqueous solution, aqueous suspension, gel, etc.

Another aspect of the disclosure provides a method for diagnosing or monitoring an ocular surface disorder in a subject. The method includes comparing the level of autoantibodies from a sample obtained from a subject with the control level of said autoantibodies to diagnosis or monitor ocular surface disorder in the subject. As used herein, the term “control level” of autoantibodies refers to the level against which autoantibodies level in the test sample can be compared including (i) those of a subject not having an ocular surface disorder, (ii) those of a subject having an ocular surface disorder, (iii) level of autoantibodies from the same subject prior to a treatment, just after starting a treatment, or prior to showing any symptoms of an ocular surface disorder.

In some embodiments, the control level can be a normal level, meaning the level in a sample from a normal patient, i.e., a subject not having an ocular surface disorder. This control level can be referred to more specifically as a “negative control”. This allows a determination based on the control level of autoantibodies, where a sample to be evaluated for an ocular surface disease has a measureable difference or substantially no difference in the autoantibodies level as compared to the control level.

In another embodiment, the control level can be the level of autoantibodies established in a sample from the subject or from a population of individuals which is believed to have an ocular surface disease. This can be more specifically referred to as a “positive control level”. The term “positive control” as used herein refers to a level of autoantibodies expression or biological activity established in a sample from a subject, from another individual, or from a population of individuals, where the sample was believed, based on data from that sample, to have the disease.

In other embodiments, the control level can be established from a previous sample from the subject being tested, so that the disease progression or regression of the subject can be monitored over time and/or the efficacy of treatment can be evaluated.

Unless otherwise stated or the context requires otherwise, the term “monitor” refers to determining the progression of an ocular surface disease or determining the effectiveness of a particular treatment protocol or a drug. The term “diagnosis” refers to a process of determining the presence or the absence of an ocular surface disease in a subject. It can also include determining which particular ocular surface disease is present in the subject.

The autoantibodies can be used for diagnosing ocular surface diseases to initiate appropriate therapy or treatment, e.g., with IgG eye drops or another appropriate pharmaceutical product.

Changes in the levels of autoantibodies can be used to assess response to or effectiveness of therapy. The treatment intensity can also be titrated to autoantibodies levels.

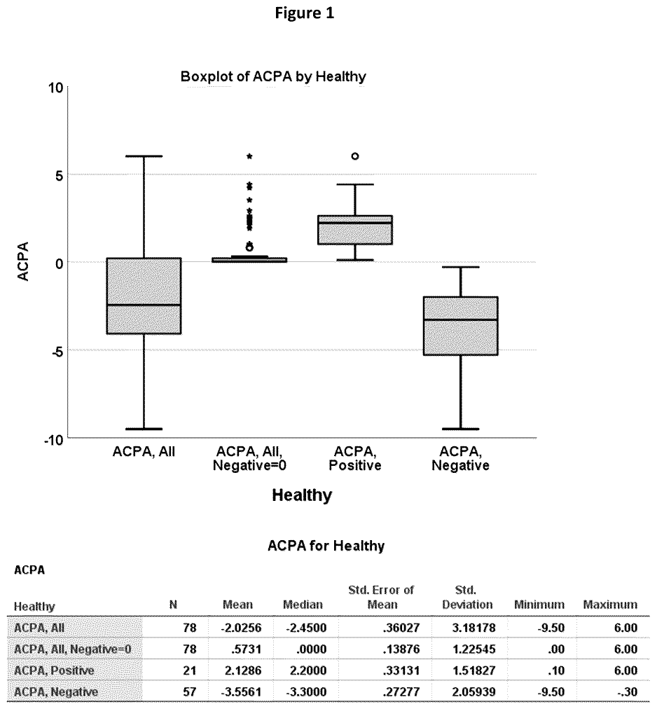

The method can be used for providing a rapid result, in-office test to diagnose and manage ocular surface diseases, and initiate/titrate IgG eye drop therapy for such diseases, either as a single agent or in combination with other active pharmacological agents. The test can be designed to measure components of tears or other ocular fluid. Concentrations of less than 5 have not been considered as remarkable to date, as discussed in relation to FIG. 1. However, levels above 5 could prove to indicate a treatable condition, and are contemplated within the scope of the present disclosure.

Treatment can occur on a periodic basis, as IgG compositions are stable for an extended period of time. Treatments can be designed for delivery at least twice per day or at least twice per month, or according to any other suitable treatment regime provided the treatment is sufficient to either reduce levels or reduce deleterious biologic actions arising from the autoantibodies present in the ocular fluid.

Methods of the disclosure utilizing the autoantibodies can also be used for diagnosing or monitoring ocular surface disorders such as oGVHD or Sjogren's syndrome and other Dry Eye Disease. Such methods typically include measuring the level of one or more autoantibodies present in a biological sample taken from a test subject (e.g., tear fluid, blood/serum, or ocular surface cells) and comparing to a control level. The control level can be prior autoantibodies level of the subject, autoantibodies level of healthy subject, or autoantibodies level of subject(s) with an ocular surface disease.

Another aspect of the disclosure provides sensors, biosensors, multi-analyte panels, arrays, assays and kits for determining the level of one or more autoantibodies obtained from the subject. The autoantibodies and methods in which they are employed can be used to assist diagnosis and to assess onset and development of ocular surface disorders. The disclosure also relates to use of autoantibodies in clinical screening, assessment of prognosis, evaluation of therapy, for drug screening and drug development.

In particular aspect, the disclosure provides a method of diagnosing or monitoring ocular surface disorders in a subject, comprising: obtaining a biological sample from the subject, and comparing the level of one or more autoantibodies in the biological sample with the control level.

The level of autoantibodies can be determined readily using any of the conventional methods available to one skilled in the art. Exemplary methods include, but are not limited to, direct or indirect methods such as coupled or uncoupled enzymatic methods, electrochemical, spectrometric (e.g., spectrophotometric such as using a UV/VIS spectrometer, fluorometric, luminometric, polarimetric, etc.) chromatographic (e.g., HPLC, gas chromatography, MPLC, LPLC, etc.), an immunological method such as ELISA, DOT-BLOT analysis etc.

Yet another aspect of the disclosure provides a method of diagnosing or monitoring ocular surface disorders, or predisposition thereto. Such a method includes comparing the level of one or more autoantibodies present in a biological sample taken from a test subject and comparing it with the control level. In one particular embodiment, the method of the disclosure is used to monitor efficacy of a therapy (e.g. a therapeutic substance) in a subject having, suspected of having, or of being predisposed to, an ocular surface disorder.

Still another aspect of the disclosure provides a multi-analyte panel or array capable of detecting one, two, three, four or more (hundreds) of autoantibodies of the disclosure. The multi-analyte panel is capable of detecting a number of different analytes. An array is capable of detecting a single analyte in a number of samples or, as a multi-analyte array, is capable of detecting a number of different analytes in a sample. A multi-analyte panel or multi-analyte array according to the disclosure is capable of detecting one or more autoantibodies as described herein, and is capable of detecting autoantibodies additional to those specifically described herein. Methods of detection may include bead-based (e.g. Luminex platform) or Phadia ImmunoCap.

Also provided is a diagnostic or monitoring test kit suitable for performing a method according to the disclosure, optionally together with instructions for use of the kit. The diagnostic or monitoring kit may comprise one or more biosensors according to the disclosure, a single sensor, or biosensor or combination of sensors and/or biosensors may be included in the kit. A diagnostic or monitoring kit may comprise a panel or an array according to the disclosure. A diagnostic or monitoring kit may comprise an assay or combination of assays according to the disclosure.

Yet further provided is the use of a method, sensor, biosensor, multi-analyte panel, array or kit according to the disclosure to identify a substance capable of modulating or treating ocular surface disorders. A substance capable of modulating or treating ocular surface disorders may be an anti-inflammatory, immunomodulatory, immunosuppressive, antibiotic substance or palliative (artificial tears, contact lenses, or punctal plugs) useful for treatment of ocular surface disorders.

Throughout this specification and the appended claims, the term “treating” or “treatment” of a disease includes: (1) preventing the disease, i.e., causing the clinical symptoms of the disease not to develop in a subject or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

A higher level of autoantibodies in the test biological sample relative to the negative control level (e.g., level of biomarker(s) in a subject who has no ocular surface disease) is indicative of the presence of an ocular surface disorder, such as, oGVHD, Sjogren's Syndrome and other Dry Eye Disease.

Methods of monitoring, determining the risk of developing and of diagnosing according to the disclosure are useful to confirm the existence of an ocular surface disorder, or predisposition thereto; to monitor development of ocular surface disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development. Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Methods for monitoring efficacy of a therapy can be used to monitor the therapeutic effectiveness of existing therapies and new therapies in human subjects and in non-human animals (e.g., in animal models). These monitoring methods can be incorporated into screens for new drug substances and combinations of substances. Modulation of a protein biomarker level is useful as an indicator of the state of the ocular surface disorder or predisposition thereto. An increase in the level of autoantibodies over time is indicative of onset or progression, i.e., worsening of the disorder, whereas a decrease in the level of protein biomarker indicates amelioration or remission of the disorder. The identification of autoantibodies for ocular surface disorders permits integration of diagnostic procedures and therapeutic regimes.

Currently there no methodologies available to determine effective treatment and it has not hitherto been possible to perform rapid assessment of drug response. Traditionally, many ocular surface disease therapies have required treatment trials lasting weeks to months for a given therapeutic approach. Detection of autoantibodies of the disclosure can be used to screen subjects prior to their participation in clinical trials. The autoantibodies provides a means to indicate therapeutic response, failure to respond, unfavorable side-effect profile, degree of medication compliance and achievement of adequate serum drug levels. The autoantibodies may be used to provide warning of adverse drug response, a major problem encountered with all ocular surface disease medications. Autoantibodies are useful in development of personalized ocular surface disease therapies, as assessment of response can be used to fine-tune dosage, minimize the number of prescribed medications, reduce the delay in attaining effective therapy and avoid adverse drug reactions. Thus by monitoring autoantibodies of the disclosure, patient care can be tailored precisely to match the needs determined by the disorder and the pharmacogenomic profile of the patient, the autoantibodies can thus be used to titrate the optimal dose, predict a positive therapeutic response and identify those patients at high risk of severe side effects.

Measurement of autoantibodies can be performed by a direct or indirect detection method. The biomarkers can be detected directly, or indirectly, via interaction with a ligand or ligands, such as an enzyme, binding receptor or transporter protein, antibody, peptide, aptamer, or oligonucleotide, or any synthetic chemical receptor or compound capable of specifically binding the biomarker. The ligand may possess a detectable label, such as a luminescent, fluorescent or radioactive label and/or an affinity tag.

In one particular embodiment, the autoantibodies of the disclosure are detected and measured using mass spectrometry-based techniques; chromatography-based techniques; enzymatic detection systems (by direct or indirect measurements); or using sensors, e.g. with sensor systems with amperometric, potentiometric, conductimetric, impedance, magnetic, optical, acoustic or thermal transducers. A sensor may incorporate a physical, chemical or biological detection system. An example of a sensor is a biosensor, i.e., a sensor with a biological recognition system, e.g., based on a nucleic acid, such as an oligonucleotide probe or aptamer, or a protein such as an enzyme, binding protein, receptor protein, transporter protein or antibody. The biosensor may incorporate an immunological method for detection of the biomarker, an electrical, thermal, magnetic, optical (e.g., hologram) or acoustic technologies. Using such biosensors, it is possible to detect the target autoantibodies at the anticipated concentrations found in biological samples. Methods of the disclosure are suitable for clinical screening, assessment of prognosis, monitoring the results of therapy, identifying patients most likely to respond to a particular therapeutic treatment, for drug screening and development, and to assist in identification of new targets for drug treatment. The identification of key autoantibodies specific to a disease is central to integration of diagnostic procedures and therapeutic regimes.

Methods involving detection and/or quantification of the autoantibodies of the disclosure can be performed using bench-top instruments, or can be incorporated onto disposable, diagnostic or monitoring platforms that can be used in a non-laboratory environment, e.g. in the physician's office or at the patient's bedside. Suitable sensors or biosensors for performing methods of the disclosure include “credit” cards with optical or acoustic readers. Sensors or biosensors can be configured to allow the data collected to be electronically transmitted to the physician for interpretation and thus can form the basis for e-medicine.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

In addition to the following examples, clinically significant results relating to the disclosure are also provided in Kwon et al., Pathological consequences of anti-citrullinated protein antibodies in tear fluid and therapeutic potential of pooled human immune globulin-eye drops in dry eye disease, Ocul Surf. 2019 Oct. 10. (pii: S1542-0124 (19) 30377-5. doi: 10.1016/j.jtos.2019.10.004) available at https://doi.org/10.1016/j.jtos.2019.10.004. Kwon et al. is incorporated herein by reference in its entirety.

### EXAMPLES

### Example 1: The Levels of Autoantibodies (ACPA) in Healthy Subjects and Ocular Surface Diseases

The presence of autoantibodies (ACPA) was demonstrated in ocular surface washings of healthy subjects (FIG. 1) to establish a cut-off for diagnosing a subject autoantibody ACPA positive. Ocular surface washings were performed in healthy subjects (no tear deficient dry eye disease (DED)) using 50 μl artificial tears and 10 μl of recovered washings were analyzed for ACPA levels by ELISA using commercial plates (CCP3). All Negative values were regarded as 0. The mean+3SD was used to establish the threshold for ACPA positive values. ACPA, All Negative=0 mean was 0.57 and 3SD 1.2=4.17. Therefore, a cut-off of 5.0 was established.

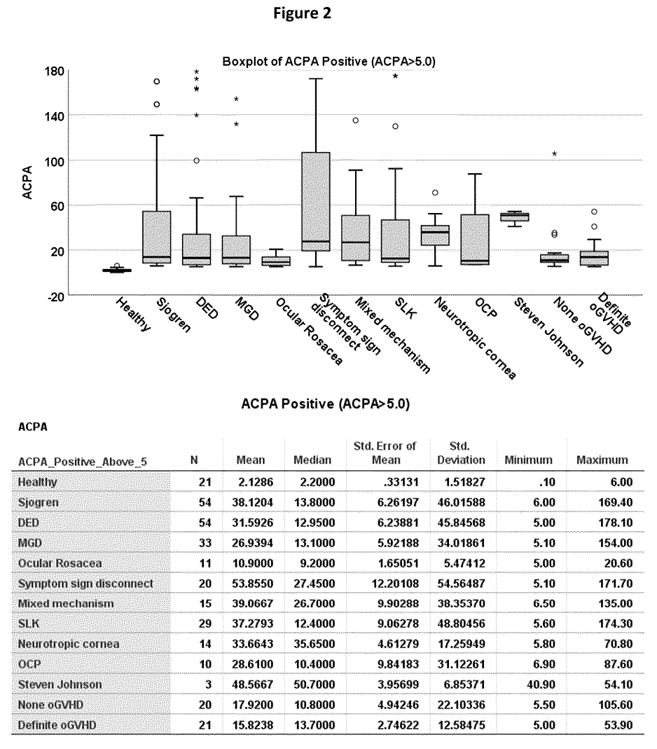

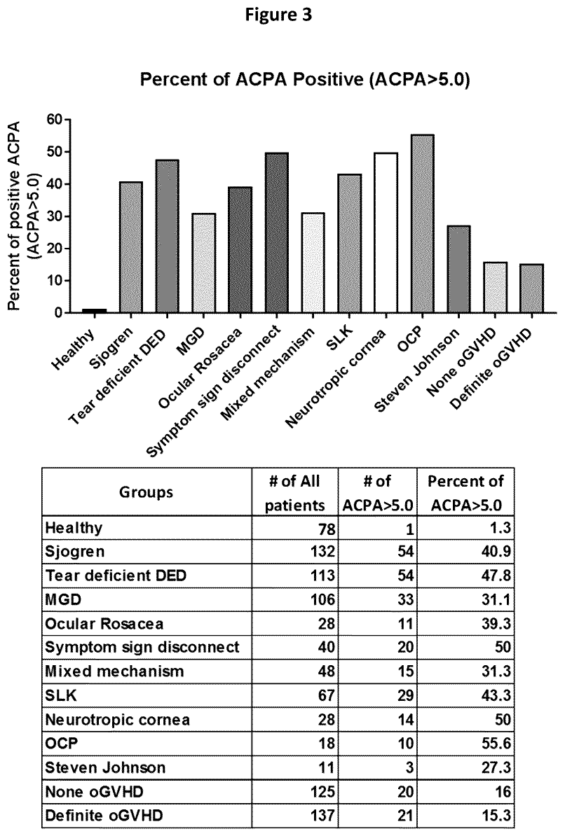

The presence of autoantibodies (ACPA) was also demonstrated in ocular surface washings of patients with ocular surface disease (FIGS. 2 and 3). As shown in FIG. 2, the levels of ACPA were determined in ocular surface washings of patients with ocular surface disease. These patients were being treated with appropriate topical eye drops (artificial tears, restasis, xiidra or steroids). Tear fluid ACPA levels were positive (i.e. ACPA>5.0) in several ocular autoimmune diseases like Sjogren syndrome, tear deficient dry eye disease, mixed mechanism DED, ocular cicatricial pemphigoid (OCP), Steven Johnson syndrome and ocular graft-versus-host disease (oGVHD). Patients with evaporative DED such as Meibomian gland dysfunction also had high ACPA levels. Superior limbic keratoconjunctivitis (SLK) and Neurotrophic keratitis also had high ACPA levels. Surprisingly, patients with symptom sign disconnect (Discordant DED) also had high ACPA levels. Thus, all of these eye diseases, including other intraocular autoimmune/inflammatory eye glaucoma would benefit from the OSIG eye drop treatment to reduce the contribution of autoantibodies to eye disease signs and symptoms. Data in FIG. 3 shows the percentage of all patients with a specific eye disease who were positive for ACPA in ocular surface washings.

### Example 2: The Inadequate Effect of Conventional Treatments on the Levels of ACPA

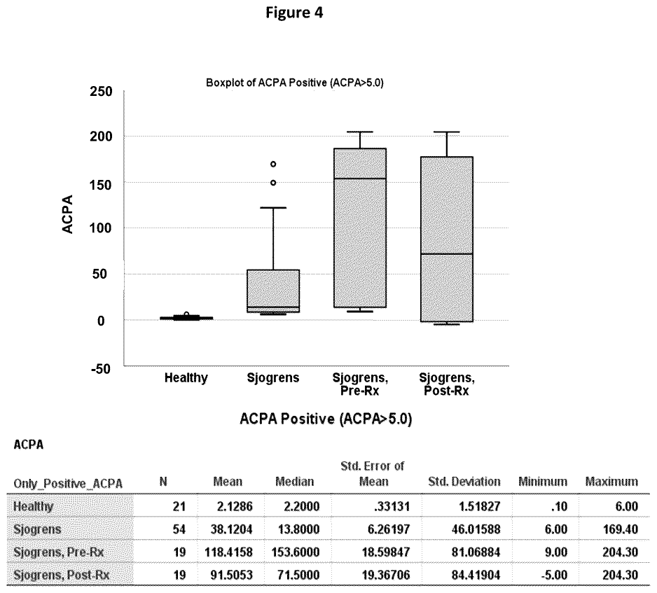

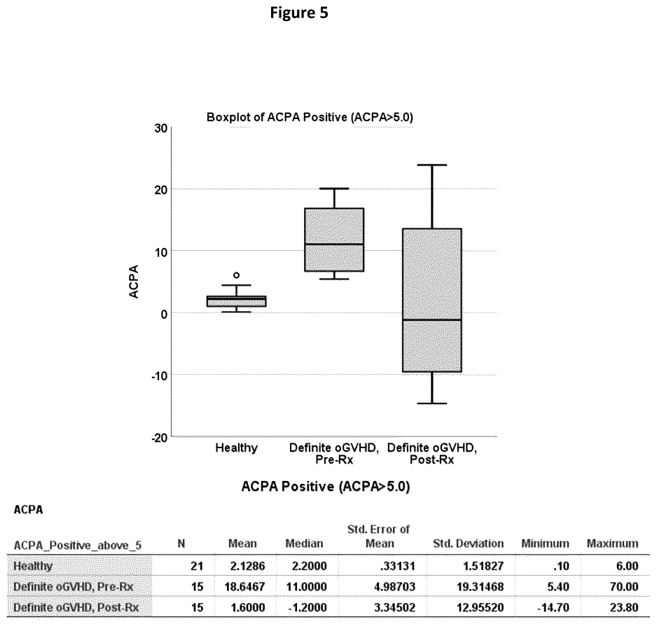

Experiments were performed to show the inadequate effect of conventional treatments on the levels of ACPA in ocular surface washings (FIGS. 4 and 5). FIG. 4 shows ACPA levels in tear fluid of Sjogren syndrome patients before and after initiation of conventional treatment. Compared to ACPA levels in Sjogren patients who are already on treatment, those patients who are not yet receiving treatment (Sjogren Pre-Rx) have significantly higher ACPA levels. After initiation of treatment (Sjogren post-Rx) levels are lower but still significantly higher as compare to health subjects. This data shows that even if Sjogren patients are getting treatment, the ACPA levels in the tear fluid are high, thus making the case for specifically targeting ACPA, such as with OSIG therapy, to reduce the contribution of ACPA to ocular surface disease.

ACPA levels in tear fluid of oGVHD of patients before and after initiation of treatment. ACPA levels in oGVHD patients who are not yet receiving treatment (oGVHD Pre-Rx) are significantly higher than ACPA levels after initiation of treatment (oGVHD post-Rx) (FIG. 5). ACP levels after one month after initiation of treatment are significantly higher than healthy subjects. This data shows that even if oGVHD patients are getting treatment, the ACPA levels in the tear fluid are high, thus making the case for specifically targeting ACPA, such as with OSIG therapy, to reduce the contribution of ACPA to ocular surface disease.

### Example 3: Citrullinated Proteins are Present on the Surface of the Eye in Patients with Ocular Surface Disease

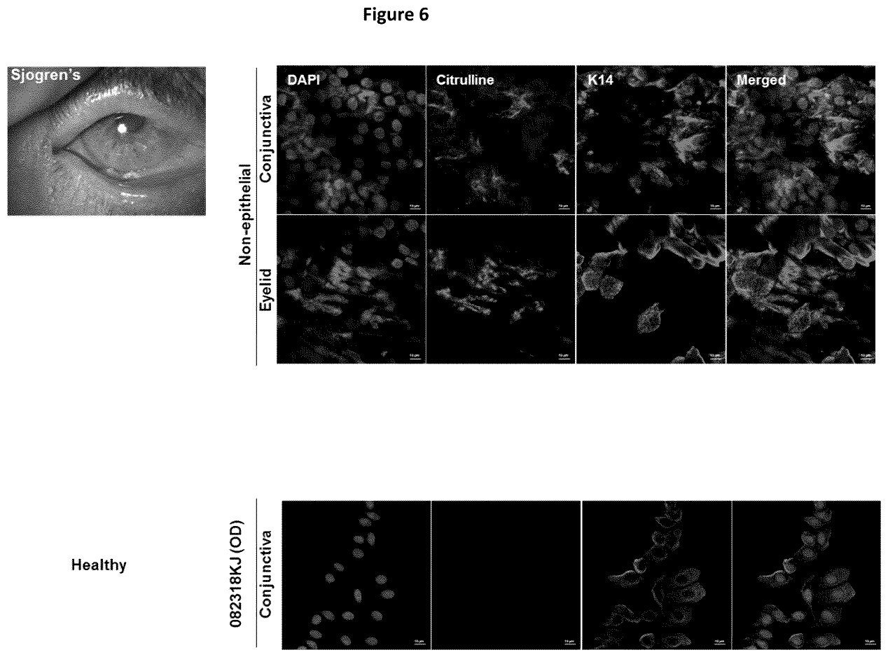

Next it was demonstrated that citrullinated proteins are present on the surface of the eye in patients with ocular surface disease. As shown in FIG. 6, citrulline are expressed on non-epithelial cells in Sjogrens-impression cytology. The purpose of this experiment was to examine whether cells on ocular surface of Sjogren's patients are citrullinated. Study approval was obtained from the Institutional Review Board of the University of Illinois at Chicago (UIC). Informed consent was obtained from all participants after the nature and possible consequences of the study were explained. Research was conducted in accordance with the requirements of the Health Insurance Portability and Accountability Act (HIPAA) and the tenets of the Declaration of Helsinki.

Antibiotic was applied on the eye of a patient, and a filter paper (PALL, #60298) was applied to temporal conjunctiva and lower eyelid to remove superficial layer of ocular surface epithelium. The filter papers were fixed with 4% paraformaldehyde (PFA) for 20 minutes in a petri dish, subsequently washed in 1×PBS for 5 minutes, and attached to a microscope slide via adhesive tab. Water resistant barrier was drawn with Pap pen around the sample, followed by 1 hour of permeabilization with PBS-T (0.025% Triton in 1×PBS). The filter papers were washed once with 1×PBS for 5 minutes with gentle shaking, and then blocked with 2.5% Donkey serum (host species secondary raised in) and 1% BSA in 1×PBS for at least 1 hour. The filter papers were then incubated with primary antibodies overnight at 4° C. Primary antibodies used were mouse monoclonal anti-citrulline antibody (1:1000; Millipore, MABN328), and rabbit polyclonal anti-cytokeratin 14 antibody (1:1000; BioLegend, #905301).

On the next day, the filter papers were washed 3 times with 1×PBS for 5 minutes with gentle shaking, followed by incubation of secondary antibodies at room temperature for 1 hour. Secondary antibodies used are as follows: Alexa Fluor 594 Donkey anti-mouse IgG (1:1000; Jackson ImmunoResearch Lab, #715-585-150) and Alexa Fluor 488 Donkey anti-rabbit IgG (1:1000; Jackson ImmunoResearch Lab, #711-546-152). After incubation, the filter papers were washed 3 times with 1×PBS for 5 minutes with gentle shaking in the dark. The slides were dried and mounted with ProLong™ Gold Antifade Mountant with DAPI (Invitrogen, P36931). A coverslip was placed and sealed with nail polish. The slides were completely dried before imaging. Images were captured using a Zeiss LSM 710 confocal microscope (Leica, UIC Ophthalmology Core facility) at 63× magnification and analyzed with the Zeiss LSM Image Software. Impression cytology was performed on Sjogren's patient, and found that citrulline was expressed on non-epithelial cells (neutrophils). Non-epithelial cells could be neutrophils, supported by presence of NETs. These data demonstrated that non-epithelial cells in Sjogren's patients were citrullinated.

### Example 4: Citrullinated Proteins are Absent in Healthy Subjects

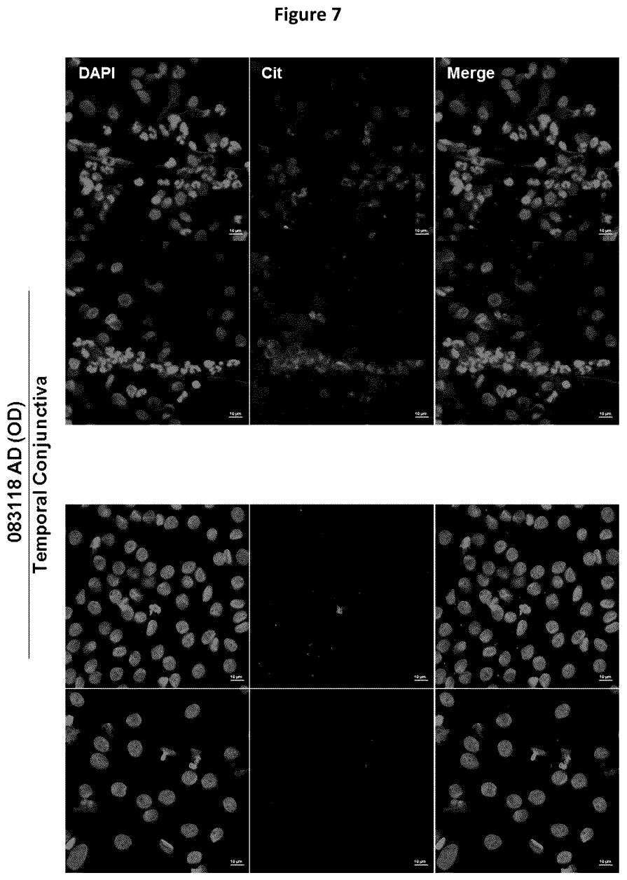

In Sjogrens, the citrullinated proteins appear to be predominantly neutrophilic (FIGS. 6 and 7). The purpose of this experiment was to verify if citrullinated non-epithelial cells are neutrophils (FIG. 7). Impression cytology was carried out as described above in Example 3.

Primary antibodies used were mouse monoclonal anti-citrulline antibody (1:1000; Millipore, MABN328), and rabbit monoclonal anti-neutrophil elstase antibody (1:500; Abcam, ab131260). On the next day, the filter papers were washed 3 times with 1×PBS for 5 minutes with gentle shaking, followed by incubation of secondary antibodies at room temperature for 1 hour. Secondary antibodies used are as follows: Alexa Fluor 594 Donkey anti-mouse IgG (1:1000; Jackson ImmunoResearch Lab, #715-585-150) and Alexa Fluor 488 Donkey anti-rabbit IgG (1:1000; Jackson ImmunoResearch Lab, #711-546-152). After incubation, the filter papers were washed 3 times with 1×PBS for 5 minutes with gentle shaking in the dark. The slides were dried and mounted with ProLong™ Gold Antifade Mountant with DAPI (Invitrogen, P36931). A coverslip was placed and sealed with nail polish. The slides were completely dried before imaging. Images were captured using a Zeiss LSM 710 confocal microscope (Leica, UIC Ophthalmology Core facility) at 63× magnification and analyzed with the Zeiss LSM Image Software.

Citrulline positive cells were neutrophils suggested by multilobed nuclei. This implies that the one major source of citrulline on the ocular surface was neutrophils. Citrullinated neutrophil proteins may be the source of ACPA response. This was validated by findings dot blot assay ACPA positive tears react with citrullinated neutrophil proteins. These data suggest neutrophils were the source of citrullination, and citrullinated neutrophil proteins may the source of ACPA response.

### Example 5: In oGVHD, the Citrullinated Proteins Appear to be Predominantly Epithelial

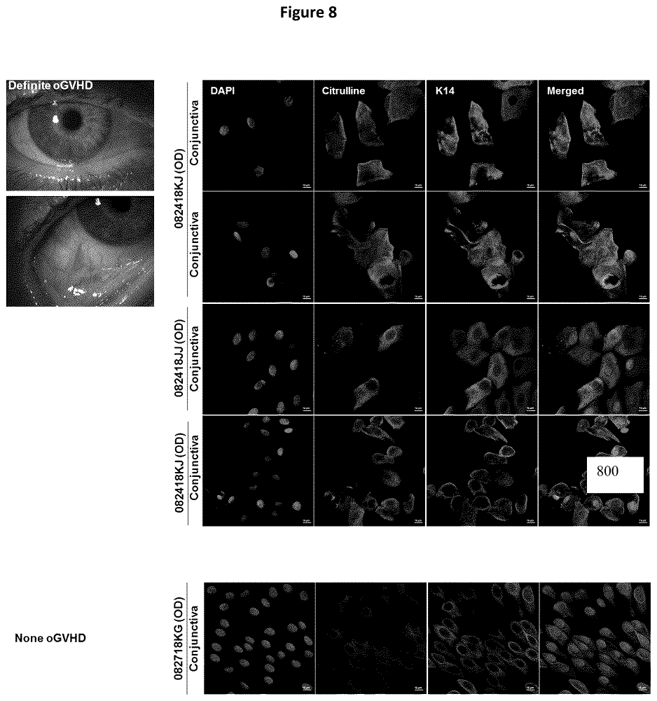

Next it was demonstrated that citrulline was expressed on epithelial cells in Definite oGVHD. The purpose of this experiment was to compare citrulline expression between Sjogrens and Definite oGVHD patients (FIG. 8). Antibiotic was applied on the eye of a patient, and a filter paper (PALL, #60298) was applied to temporal conjunctiva and lower eyelid to remove superficial layer of ocular surface epithelium. The filter papers were fixed with 4% paraformaldehyde (PFA) for 20 minutes in a petri dish. They were then washed in 1×PBS for 5 minutes, and attached to a microscope slide via adhesive tab. Water resistant barrier was drawn with Pap pen around the sample, followed by 1 hour of permeabilization with PBS-T (0.025% Triton in 1×PBS). The filter papers were washed once with 1×PBS for 5 minutes with gentle shaking, and then blocked with 2.5% Donkey serum (host species secondary raised in) and 1% BSA in 1×PBS for at least 1 hour. The filter papers were then incubated with primary antibodies overnight at 4° C. Primary antibodies used were mouse monoclonal anti-citrulline antibody (1:1000; Millipore, MABN328), and rabbit polyclonal anti-cytokeratin 14 antibody (1:1000; BioLegend, #905301). On the next day, the filter papers were washed 3 times with 1×PBS for 5 minutes with gentle shaking, followed by incubation of secondary antibodies at room temperature for 1 hour. Secondary antibodies used are as follows: Alexa Fluor 594 Donkey anti-mouse IgG (1:1000; Jackson ImmunoResearch Lab, #715-585-150) and Alexa Fluor 488 Donkey anti-rabbit IgG (1:1000; Jackson ImmunoResearch Lab, #711-546-152).

After incubation, the filter papers were washed 3 times with 1×PBS for 5 minutes with gentle shaking in the dark. The slides were dried and mounted with ProLong™ Gold Antifade Mountant with DAPI (Invitrogen, P36931). A coverslip was placed and sealed with nail polish. The slides were completely dried before imaging. Images were captured using a Zeiss LSM 710 confocal microscope (Leica, UIC Ophthalmology Core facility) at 63× magnification and analyzed with the Zeiss LSM Image Software. Unlike Sjogren's where neutrophils were the major source of citrulline, Definite oGVHD patients have whole epithelial cells citrullinated, which correlates with high level of citrulline and PAD4 enzyme. These data showed that strong citrullination of epithelial cells supports the high level of citrulline and PAD4 enzyme. Of the patients tested, there was no colocalization of citrulline and K14 in two different Sjogrens patients, but there was colocalization of citrulline and K14 in oGVHD patients. In addition, co-localization of citrulline with neutrophils was observed in two different Sjogrens patients.

### Example 6: Inducible Fc Receptor Expressed on Neutrophils

Citrullinated proteins were absent in patients with none-oGVHD. These findings demonstrate that one source of autoantibody production are citrullinated neutrophils and/or ocular surface cells in DED patients. It was also demonstrated that Fc receptors (receptors for IgG) are present on the surface of the eye, and the ACPA antibodies interact with Fc receptors to produce surface diseases. It is hypothesized that Fc receptors are not expressed on naïve neutrophils but are inducible. Fc receptors are expressed in neutrophils within mucocellular aggregates.

The purpose of this experiment was to support the hypothesis of inducible Fc receptor on neutrophils. Peripheral blood was collected by venipuncture in BD vacutainer sodium heparin tubes (BD Biosciences, #367878) and immediately transported to the laboratory for neutrophil isolation. Neutrophils were isolated by immunomagnetic depletion of non-target cells using MACSxpress beads (MACSxpress neutrophil isolation kit, Miltenyi Biotech, #130-104-434) according to the manufacturer's instruction. The residual erythrocytes were removed using MACSxpress erythrocyte depletion kit (Miltenyi Biotec, #130-094-183). Isolated neutrophils were resuspended with 3 mL of serum free phenol red free RPMI-1640 medium (GIBCO, #11835-030). After the measurement of cell numbers, 50,000 cells/200 μL of neutrophils were loaded into an EZ single Cytofunnel (Thermo Scientific, #A78710003). Patient's mucoid sample was prepared with 4 U/mL DNaseI and 1× Buffer in 100 μL solution, and incubated for 30 minutes at 37° C. to achieve single cell suspension. 10 fold diluted mucoid sample was loaded into an EZ single Cytofunnel (Thermo Scientific, #A78710003). The samples were centrifuged at 1000 rpm for 5 min by Cytospin 4 (Thermo Scientific, Kalamazoo, MI) to achieve a monolayer of cell deposition in a defined area of the cytoside (Thermo Scientific, #5991056). After centrifugation, slides were air dried for 5 min and then fixed with 4% paraformaldehyde (PFA) (Electron Microscopy Sciences, Hatfield, PA, #15710) solution for 20 minutes. Water resistant barrier was drawn with Pap pen around the sample, followed by 1 hour of permeabilization with PBS-T (0.025% Triton in 1×PBS). The samples were washed once with 1×PBS for 5 minutes with gentle shaking, and then blocked with 2.5% Donkey serum (host species secondary raised in) and 1% BSA in 1×PBS for at least 1 hour. The samples were then incubated with primary anti-bodies overnight at 4° C. Primary antibodies used were mouse monoclonal anti-CD64 antibody (1:1000; Santa Cruz, sc-1184), mouse monoclonal anti-IgMK antibody (1:2500; Novus Biologicals, NBP1-96975), rabbit monoclonal anti-neutrophil elstase antibody (1:500; Abcam, ab131260) and rabbit monoclonal IgG antibody (0.03 μg/mL; Abcam, ab172730).

On the next day, the samples were washed 3 times with 1×PBS for 5 minutes with gentle shaking, followed by incubation of secondary antibodies at room temperature for 1 hour. Secondary antibodies used are as follows: Alexa Fluor 594 Donkey anti-mouse IgG (1:1000; Jackson ImmunoResearch Lab, #715-585-150) and Alexa Fluor 488 Donkey anti-rabbit IgG (1:1000; Jackson ImmunoResearch Lab, #711-546-152). After incubation, the samples are washed 3 times with 1×PBS for 5 minutes with gentle shaking in the dark. The slides were dried and mounted with ProLong™ Gold Antifade Mountant with DAPI (Invitrogen, P36931). A coverslip was placed and sealed with nail polish. The slides were completely dried before imaging. Images were captured using a Zeiss LSM 710 confocal microscope (Leica, UIC Ophthalmology Core facility) at 63× magnification and analyzed with the Zeiss LSM Image Software.

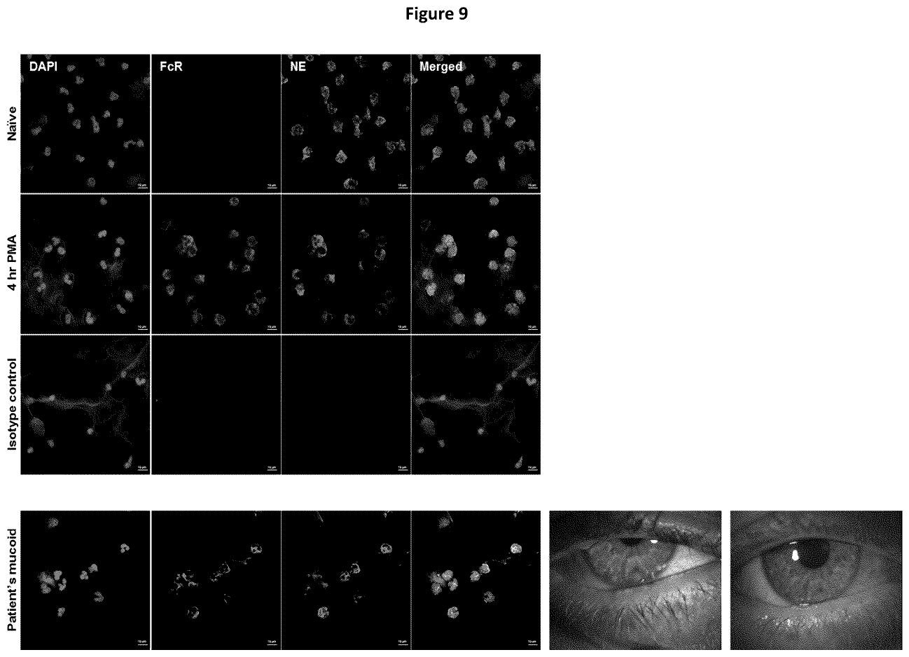

As shown in FIG. 9, Fc receptors are expressed only on PMA stimulated neutrophils and patient's mucoid, which implies that neutrophils have been activated. The staining was confirmed positive with isotype control and negative control. In summary, naïve neutrophils do not express Fc receptor, while activated neutrophils do, which confirms that expression of Fc receptors are inducible.

As additional experiment was carried out to confirm the presence of Fc receptor positive neutrophils on ocular surface.). Impression cytology was carried out as described above in Example 3. Primary antibodies used were mouse monoclonal anti-CD64 antibody (1:1000; Santa Cruz, sc-1184), and rabbit polyclonal anti-cytokeratin 14 antibody (1:1000; BioLegend, #905301). On the next day, the filter papers were washed 3 times with 1×PBS for 5 minutes with gentle shaking, followed by incubation of secondary antibodies at room temperature for 1 hour. Secondary antibodies used are as follows: Alexa Fluor 594 Donkey anti-mouse IgG (1:1000; Jackson ImmunoResearch Lab, #715-585-150) and Alexa Fluor 488 Donkey anti-rabbit IgG (1:1000; Jackson ImmunoResearch Lab, #711-546-152). After incubation, the filter papers were washed 3 times with 1×PBS for 5 minutes with gentle shaking in the dark. The slides were dried and mounted with ProLong™ Gold Antifade Mountant with DAPI (Invitrogen, P36931). A coverslip is placed and sealed with nail polish. The slides were completely dried before imaging. Images are captured using a Zeiss LSM 710 confocal microscope (Leica, UIC Ophthalmology Core facility) at 63× magnification and analyzed with the Zeiss LSM Image Software. After immunofluorescence staining, the same filter papers were Hematoxylin and Eosin stained. Filter papers were stained with hematoxylin (Fisher Scientific, SH26-500D), rinsed in acid, dipped in bluing solution, and counterstained with eosin (Thermo Scientific, Waltham, MA). Slides were examined using an upright Axioscope 100 microscope (Carl Zeiss Meditec GmbH, Hamburg, Germany), imaged using a Zeiss MRc color camera, and analyzed using Zeiss Axiovision.

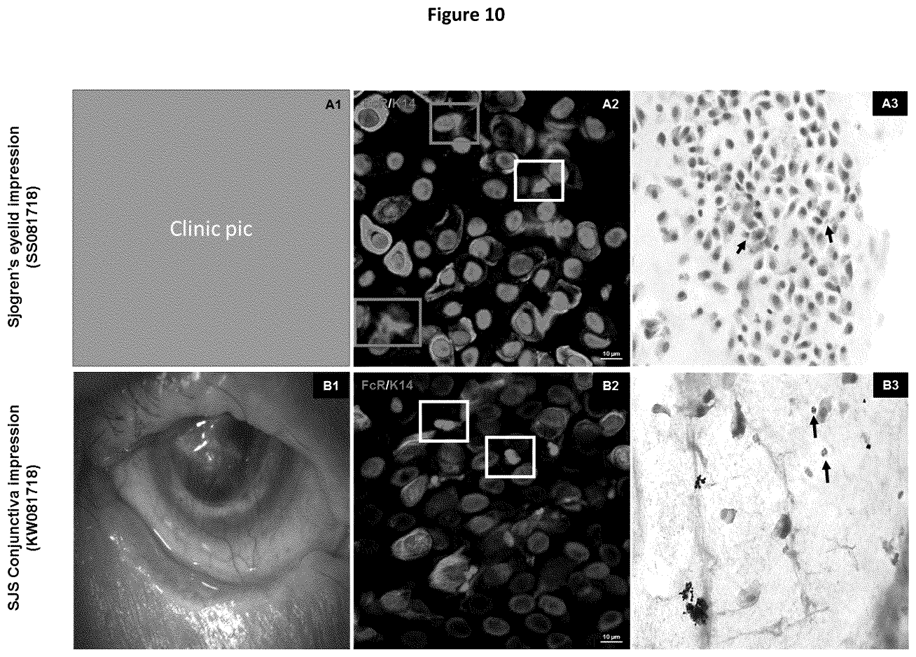

FIG. 10 demonstrates that Fc receptors were expressed on K14 negative cells (non-epithelial cells), intermixed with epithelial cells. Some of these non-epithelial cells have multi-lobed nuclei (white box) which is reminiscent of neutrophils, which suggest neutrophils, and is further confirmed by H&E staining. Other cells are large mononuclear cells which could be T-cells. K14 positive cells do not show Fc receptors, which is confirmed by the literature.

In addition, an experiment was carried out to determine if ACPA in tears is reactive to NET-citrullinated proteins. Dot blot assay was performed using Bio-Dot Microfiltration Apparatus (Bio-Rad, #170-6545), with a nitrocellulose membrane (Company, cat #). Overall protocol was followed by manufacturer's instructions. The membrane was pre-wetted in TBS; It was re-hydrated as necessary. 1 μg/100 μL of total protein from lysate was applied to the membrane, incubated for an hour, with vacuum chamber open to air, allowing protein to filter through the membrane by gravity. 200 μL of blocking solution (1% BSA-TBS) was added to each well, and let it filter through by gravity, followed by washing. 200 μL of wash solutions (0.05% Tween-TBS) was added and vacuumed out, and repeated one more time. 100 μL 1:80 diluted patient's tears (diluted in 1% BSA-TTBS) was added and let filter through by gravity, followed by washing the membrane with 200 μL of TTBS. The membrane was incubated with 100 μL of HRP-conjugated human IgG antibody (#A112P, EMD). The blots were incubated with enhanced chemiluminescence SuperSignal West Femto maximum sensitivity substrate (34095, Thermo Fisher) and the signal was detected with ImageQuant LAS 4000 system (GE Lifesciences Inc). The intensity of dot blots were determined with Image J software.

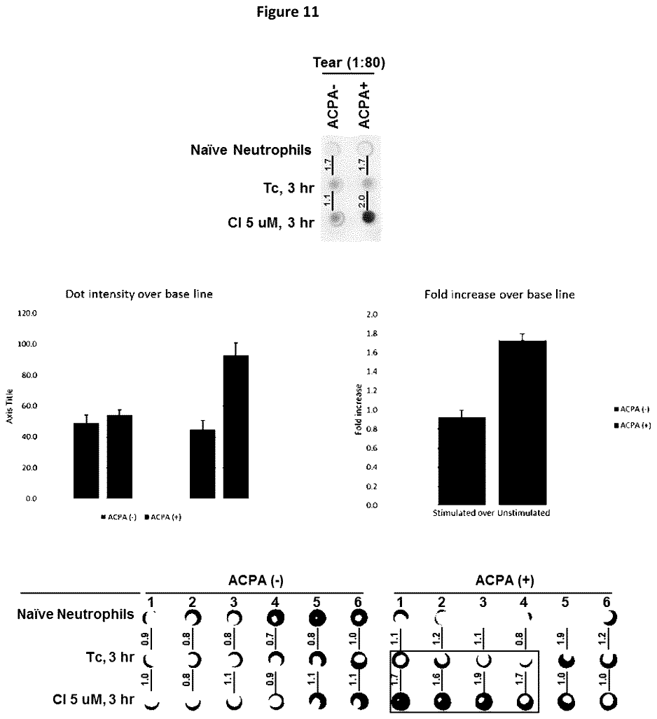

Neutrophils were simulated with calcium ionophore for 3 hours as it induces hyper-citrullination and RPMI for the same period of time, which served as a control. After stimulation, neutrophils were lysed to extract total protein, which are immobilized to the membrane. As shown in FIG. 11, the ACPA in tears were found to react with NET-proteins, confirming that neutrophilic citrullinated proteins may be one source of ACPA stimulation. The results were expressed as fold ratio was calculated by using the following formula: Fold=intensity of citrullinated protein/intensity of wild-type protein. ACPA positive tears show a greater reactivity to stimulated NET-proteins (92.8±7.85) than unstimulated ones (54.1±5.46), whereas ACPA negative tears did not show a difference (45.0±3.19 vs. 49.3±4.79). In summary, ACPA in tears is reactive to NET-proteins.

Another experiment was carried out to determine if polyclonal ACPA were present in tears. Dot blot assay was performed using the Direct Detect assay free cards (#DDAC00010-GR) were obtained from Millipore (Burlington, MA). A 2 μL of recombinant protein (1 μg) was applied to the membranes and allowed to dry at room temperature (RT) for 20 min. Then the membranes were blocked with 10% BSA and 0.05 Tween-20 prepared in 1×PBS (pH 7.4) for 30 min at room temperature. The membranes were rinsed with rinsing buffer (1% BSA and 0.05% Tween 20 in 1×PBS) and probed with the patients tear sample (dilution 1:40, diluted with sterile water) were applied to the membranes and incubated for 30 min at RT. Membranes were washed thrice with rinsing buffer and subsequently incubated with HRP-conjugated human IgG antibody (#A112P, EMD) for 30 min at RT. Membranes were washed thrice with rinsing buffer. The blots were incubated with enhanced chemiluminescence SuperSignal West Femto maximum sensitivity substrate (34095, Thermo Fisher) and the signal was detected with ImageQuant LAS 4000 system (GE Lifesciences Inc). The intensity of dot blots were determined with Image J software. The results shown in Table 2 are expressed as fold ratio, as calculated using the following formula: Fold=intensity of citrullinated protein/intensity of wild-type protein (Table 2).

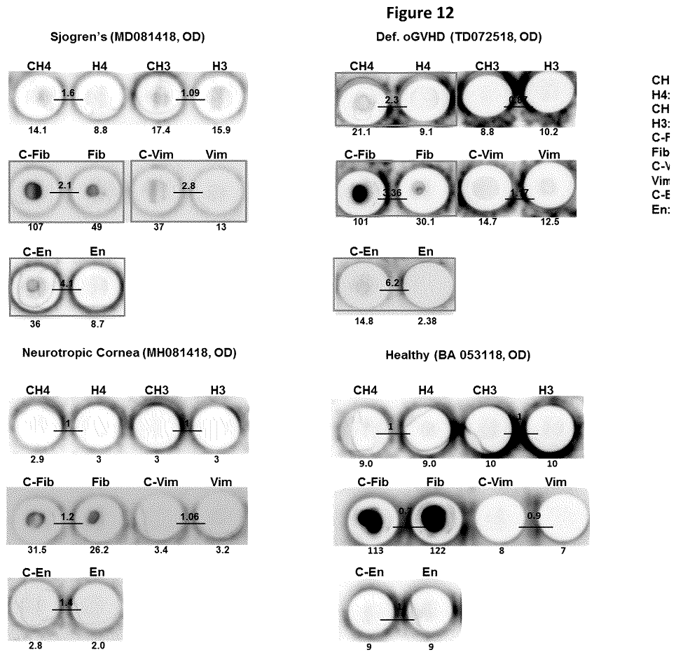

As shown in FIG. 12, the ACPA positive tears show the presence of polyclonality, and the ACPA react with several citrullinated proteins. The fold change of citrullinated fibrinogen or enolase over its wild type was greater than 1.5 in both Sjogren's patients and Definite oGVHD patients AS. In Sjogrens patients, citrullinated vimentin was greater than 1.5 fold change over its wild type, while in Definite oGVHD, citrullinated Histone H4 was greater than wild type. Tears from two diseased patient demonstrated the polyclonality of ACPA in their tears, while neurotropic and healthy tears are negative for ACPA. In summary, ACPA positive tears shows polyclonality. (+): >1.5.

TABLE 2
Fold change of citrullinated protein over wild type protein

| | Fold change of citrullinated protein | | | | |
|---|---|---|---|---|---|
| | over wild type protein | | | | |
| | Histone | Histone | | | |
| | H4 | H3 | Fibrinogen | Vimentin | Enolase |
| Neurotropic | 1 | 1 | 1.2 | 1.06 | 1.4 |
| Healthy | 1 | 1 | 0.7 | 0.9 | 1 |
| Sjogrens | 1.6 | 1.09 | 2.1 | 2.8 | 4.1 |
| Definite | 2.3 | 0.8 | 3.36 | 1.17 | 6.2 |
| oGVHD | | | | | |

### Example 7: ACPA H4R3Cit Antiserum in Ocular Surface Disease

Next, it was examined whether a specific autoantibody-ACPA (H4R3cit antiserum)—causes ocular surface disease in an experimental model. Specifically, this experiment investigates whether f H4R3cit was present on patient's mucocellular aggregates. Mucocellular aggregates (MCA) were collected from patient eyes using a sterile jeweler's forceps and transferred to a sterile 0.2 mL PCR tubes containing Refresh Optive and stored in an ice box. Fresh MCA samples were embedded in Tissue-Tek® OCT compound (Sakura Finetek, Torrance, CA, #4583) and flash frozen. Frozen sections were cut at 10 μm thickness using a cryostat (Thermo Scientific, CryoStar NX50, #957130). This particular MCA was collected from a patient who was diagnosed with Definite oGHVD. For immunostaining, the slides were air dried, then fixed with 4% PFA for 20 minutes. The samples were then permeabilized with 0.025% Triton X-100 (Fisher Scientific, #BP151-100) and blocked with freshly prepared 10% Donkey serum with 1% Bovine Serum Albumin (Gemini Bio-Products, #700-100P) for 2 hours. The samples were incubated with primary antibodies overnight at 4° C. The following primary antibodies are used: mouse monoclonal anti-human neutrophil elastase (NE) (1:100; Dako, #M0752), rabbit polyclonal anti-Histone H4 (citrulline R3) antiserum (1:1000; Abcam, ab81797) and rabbit polyclonal IgG (1:1000; Abcam, ab37415). The slides were washed three times with 1×PBS for 5 minutes with gentle shaking, and incubated with secondary antibodies diluted in blocking buffer at room temperature for 1.5 hours. Secondary antibodies used were as follows: Alexa Fluor 594 Donkey anti-mouse IgG (1:1000; Jackson Immuno Research Lab, #715-585-150) and Alexa Fluor 488 Donkey anti-rabbit IgG (1:1000; Jackson ImmunoResearch Lab, #711-546-152). The slides were washed twice with 1×PBS, counterstained with Hoechst 33342 (2 μl/mL) (Thermo Scientific, #62249) for 10 min, a quick PBS wash and covered with a drop of ProLong™ Gold antifade reagent (Invitrogen, #P36930). Images were captured using a Zeiss LSM 710 confocal microscope (Leica, UIC Ophthalmology Core facility) at 100× magnification and analyzed with the Zeiss LSM Image Software.

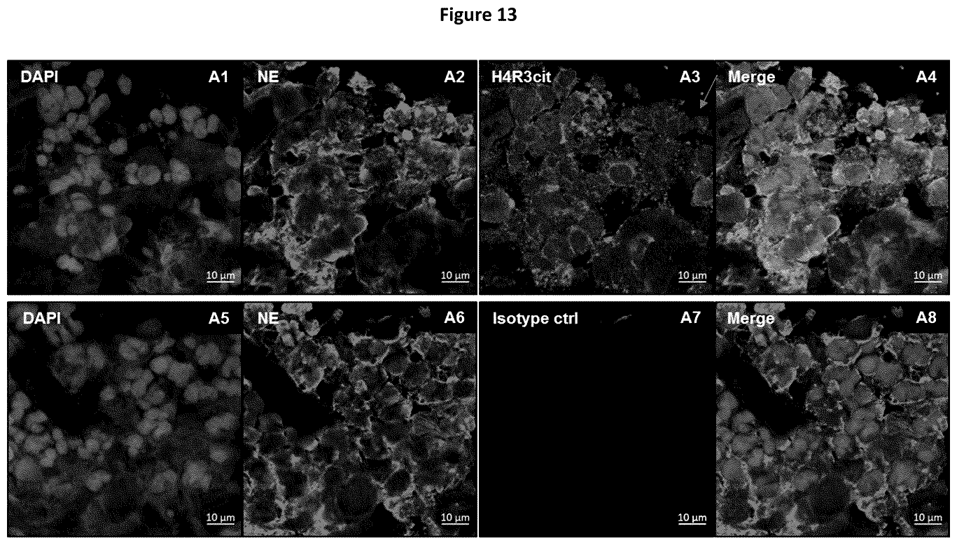

To confirm the presence of H4R3cit on ocular surface of the patient, MCA was collected from a patient who was diagnosed with Definite oGVHD, and analyzed with immunofluorescence staining. Immunofluorescence stained cells showed a large number of neutrophils with multi-lobed nuclei and they were confirmed with positive staining of neutrophil elastase. It is known in the art that, H4R3cit is expressed around the nucleus (perinuclear), which can be seen in FIG. 6. As shown in FIG. 13 H4R3cit at H immunolocalized over the ocular surface and dot blot analysis using oGVHD patient's tears show reaction with H4cit. FIG. 13 also shows the presence of H4R3cit on patient's mucocellular aggregates. The staining was confirmed positive, as isotype control showed negative staining. In summary, patient's mucocellular aggregate has H4R3cit antigen, which may be the source of ACPA response.

In order to investigate whether ACPA antibodies cause ocular surface disease, Thy1-YFP mice at the age of 8-10 weeks were used for treatment experiment. Thy1-YFP mice (n=5/group) were anesthetized by intraperitoneal injection of a combination of ketamine (20 mg/kg; Phoenix Scientific, St. Joseph, MO) and Xylazine (6 mg/kg; Phoenix Scientific). To assess the pathologic effects of ACPA, ACPA antibodies were applied to the mouse cornea and determined the extent of cornea surface disease induced by the antibodies and compared them with application of non-ACPA anti-bodies. The following conditions were used: 1) Non-ACPA antibody: Anti-Histone H4 antibody (100 ng/ml; Cell Signaling #2592, 2) ACPA antibody #1: anti-Histone H4 (citrulline R3) (H4R3cit) antiserum (100 ng/mL; Abcam #ab81797), 3) ACPA antibody #2: anti-Histone H3 (citrulline R2+R8+R17) antiserum (100 ng/ml; Novus Biologicals #NB100-57135SS) was applied on the surface of the murine cornea (10 μL/cornea) and incubated for 40 minutes for 7 consecutive days. Since ACPA antibodies used were in rabbit antiserum, normal rabbit serum application (100 ng/ml; Abcam #ab7487) was used as a control. Dilutions for all antibodies were made in Refresh Optive. For fluorescein staining experiment, 10 μl of 0.2% fluorescein was instilled on mice cornea for 1 minute and washed two times with 1×PBS. The fluorescein staining was detected and photographed with a slit lamp (Haag Streit, Bern, Switzerland), using cobalt blue illumination and a barrier yellow filter. Images from each group (n=5) were collected and the intensity of fluorescein staining was measured by Metamorph image system. (Metamorph, Universal Imaging, Downington, PA).

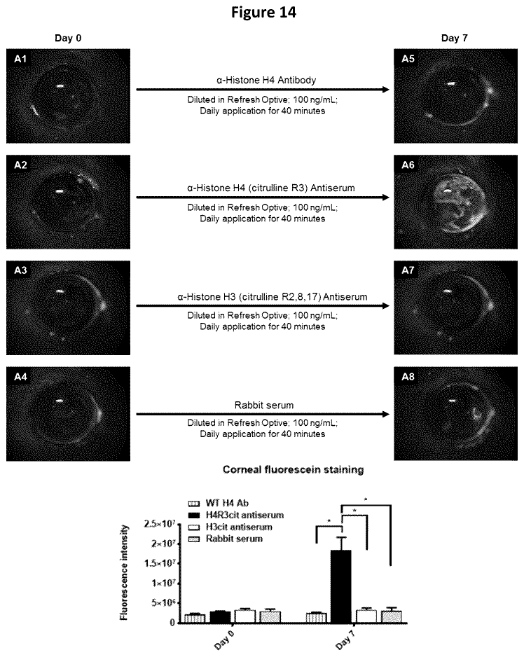

H4R3cit antiserum was applied over mouse cornea demonstrated a significant increase in ocular surface disease as evidenced by fluorescein staining of the cornea. The fluorescein staining was significantly increased in several controls (Rabbit serum, H3cit antiserum and wild type H4 antibody) (FIG. 14). As shown in FIG. 14, panels A1 and A5 show that non-ACPA antibody application for 7 consecutive days on mouse cornea did not result in corneal epithelial disease, as evidenced by lack of fluorescein staining on day 7 (2.59×10⁶±5.41×10⁴). Of the two ACPA antibodies used, only one resulted in corneal epithelial disease as evidenced by significant increase in corneal fluorescein staining on day 7. The ACPA antibody that caused corneal epithelial disease was ACPA #1-H4R3cit antiserum (fluorescence intensity of 1.85×10⁷±3.19×10⁶ on day 7 compared to day 0 (3.00×10⁶±6.05×10⁴; p=0.0006; FIG. 14. A2 and A6), whereas ACPA #2-H3cit antiserum did not cause corneal epithelial disease (fluorescence intensity of 3.34×10⁶±4.90×10⁵ on day 7 compared to day 0 (3.36×10⁶±3.05×10⁵; p=0.97; FIG. 14. A3 and A7). The vehicle control also did not cause corneal epithelial disease, as evidenced by lack of fluorescein staining on Day 7 (3.11×10⁶±3.49×10⁵; FIG. 14. A4 and A8). In summary, these data show that ACPA antibodies caused corneal epithelial disease, however not all ACPA antibodies can do so.

TABLE 3
Fluorescence measurements from fluorescein staining of mouse cornea

| | Day 0 Fluorescence | Day 7 Fluorescence | P value |
|---|---|---|---|
| Conditions | (Mean ± SEM, AU) | (Mean ± SEM, AU) | (two-tailed) |
| Non-ACPA | 2.22 × 10⁶ ± 1.04 × 10⁵ | 2.59 × 10⁶ ± 5.41 × 10⁴ | 0.013 |
| (WT H4 antibody) | | | |
| ACPA #1 | 3.00 × 10⁶ ± 6.05 × 10⁴ | 1.85 × 10⁷ ± 3.19 × 10⁶ | 0.0006 |
| (H4R3cit antiserum) | | | |
| ACPA #2 | 3.36 × 10⁶ ± 3.05 × 10⁵ | 3.34 × 10⁶ ± 4.90 × 10⁵ | 0.97 |
| (H3cit antiserum) | | | |
| Control | 3.04 × 10⁶ ± 2.14 × 10⁵ | 3.11 × 10⁶ ± 3.49 × 10⁵ | 0.86 |
| (Rabbit serum) | | | |

An experiment was also performed to determine whether ACPA antibodies could induce NETosis. Thy1-YFP mice at the age of 8-10 weeks were used for treatment experiment. Thy1-YFP mice (n=5/group) were anesthetized by intraperitoneal injection of a combination of ketamine (20 mg/kg; Phoenix Scientific, St. Joseph, MO) and Xylazine (6 mg/kg; Phoenix Scientific). Mice were divided into three groups: 1) RPMI (Gibco #11835030)-treated, which served as a control, 2) ACPA antibody #1: H4R3cit antiserum or 3) ACPA antibody #2: H3cit antiserum-treated. 10 μL of each treatment was applied on murine corneas. The eyes were treated for 7 consecutive days. At the end of experiment, a filter paper staining was used to perform impression cytology of the murine cornea. On a microscope slide, an adhesive tab (EMS, #76760) was attached. A filter paper (Millipore, #JHWP02500) was cut into an optimal size. While a mouse was under anesthesia, a drop of proparacaine was applied on mouse eye for one minute and gently removed with Kimwipe. A piece of filter paper was placed on the cornea and pressed gently for 30 seconds. Then it was gently lifted up and placed on the adhesive tap, touched area facing up. All the following steps were done in a humid chamber, except washing steps. Water resistant barrier was drawn with Pap pen, and the filter paper was fixed with 4% paraformaldehyde (PFA) for 20 minutes. The filter papers were washed with 1×PBS for 5 minutes with gentle shaking, followed by 10 minutes of permeabilization with PBS-T (0.025% Triton in 1×PBS). The filter papers were washed once with 1×PBS for 5 minutes with gentle shaking, and then blocked with 2.5% Donkey serum (host species secondary raised in) and 1% BSA in 1×PBS for at least 1 hour. The filter papers were then incubated with primary antibodies overnight at 4° C. Primary antibodies used were mouse monoclonal anti-citrulline antibody (1:1000; Millipore, MABN328), and rabbit polyclonal anti-Histone H4 (citrulline R3) antiserum (1:1000; Abcam, ab81797). On the next day, the filter papers were washed 3 times with 1×PBS for 5 minutes with gentle shaking, followed by incubation of secondary antibodies at room temperature for 1 hour. Secondary antibodies used are as follows: Alexa Fluor 594 Donkey anti-mouse IgG (1:1000; Jackson Immu-noResearch Lab, #715-585-150), Alexa Fluor 488 Donkey anti-rabbit IgG (1:1000; Jackson Immu-noResearch Lab, #711-546-152), Alexa Fluor 594 Donkey anti-rabbit IgG (1:1000; Invitrogen, #A21207), and Alexa Fluor 488 Donkey anti-mouse IgG (1:1000; Jackson ImmunoResearch Lab, #715-547-003). After incubation, the filter papers were washed 3 times with 1×PBS for 5 minutes with gentle shaking in the dark. The slides were dried and mounted with ProLong™ Gold Antifade Mountant with DAPI (Invitogen, P36931). A coverslip was placed and sealed with nail polish. The slides were completely dried before imaging. Images were captured using a Zeiss LSM 710 confocal microscope (Leica, UIC Ophthalmology Core facility) at 63× magnification and analyzed with the Zeiss LSM Image Software. After 7 days of treatment with 1) RPMI, 2) ACPA #2-H3cit antiserum, or 3) ACPA #1-H4R3cit anti-serum, mouse cornea epithelium was lifted up on a filter paper to investigate other pathological effects.

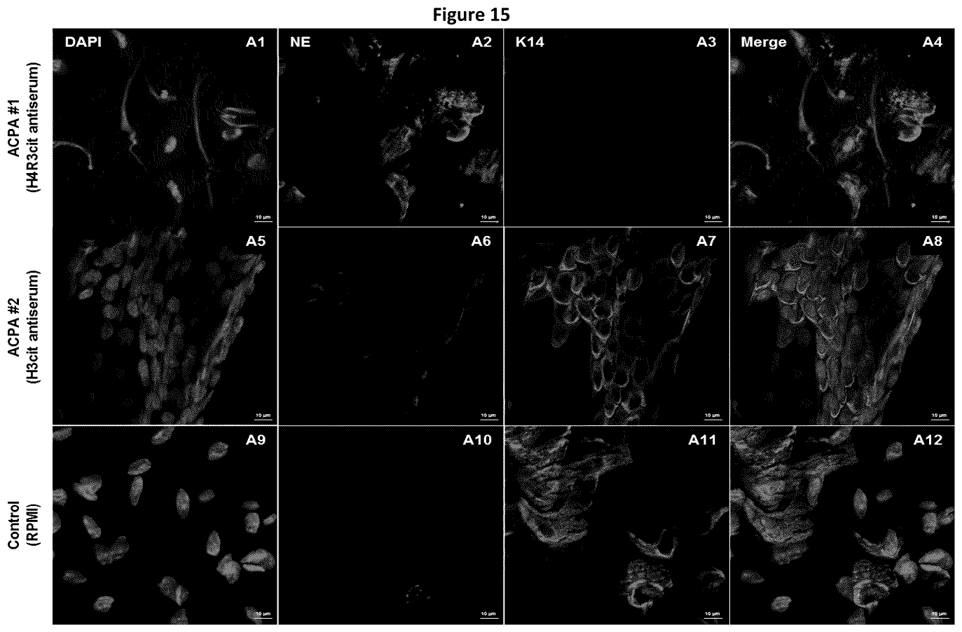

As shown in FIG. 15, NETosis was induced by ACPA-H4R3cit antiserum. Presence of neutrophils and Neutrophil Extracellular Traps (NETs) were observed only in the ACPA #1-H4R3cit antiserum treated group (FIG. 15. A1); NETs were confirmed with neutrophil elastase staining (FIG. 15. A2). This suggests that H4R3cit antiserum induced NETosis. In summary, ACPA antibody #1 caused NETosis and induced citrullination on mouse cornea epithelium. This data suggests that ACPA antibodies contribute toward a feed-forward cycle of citrullination to further amplify citrullination related eye disease.

### Example 8: Blocking Interaction of ACPA (H4R3Cit)—with Fc Receptor Blocking Strategies Abrogated the Ocular Surface Disease Induced by ACPA-H4R3Cit

In order to determine whether ACPA pathological effect on cornea is mediated via Fc receptors, Thy1-YFP mice at the age of 8-10 weeks were used for treatment experiment. A peptide that blocks all Fc receptors was used and then applied the ACPA antibody. Using 10 μL of 1) Azide-free Fc receptor blocker (Innovex Biosciences #NB355) or 2) Scrambled peptide, and applied on murine cornea and incubated for 30 minutes. This pre-incubation with peptides was performed under no anesthesia. Then, Thy1-YFP mice (n=5/group) were anesthetized, and 10 μL of ACPA #1-H4R3cit antiserum was applied for both Fc receptor blocked and scrambled peptide blocked group, and incubated for 40 minutes for 10 consecutive days. Dilutions were made in Refresh Optive. Murine corneas were fluorescein stained, monitored and imaged with a slit lamp. The fluorescein intensity was measured by Metamorph image system. To investigate if competitive blocking of Fc receptor would reproduce the same results as peptide-blocking of Fc receptor, mouse IgG was added along with ACPA antibody. For IgG competition experiment, 10 μL of mixture of ACPA antibody (H4R3cit antiserum) and mouse IgG (Abcam #ab188776) in 1:1 ratio was applied on a murine cornea and incubated for 40 minutes for 10 consecutive days. In control experiment, only ACPA antibody was used. Murine corneas were fluorescein stained, monitored and imaged with a slit lamp. The fluorescein intensity was measured by Metamorph image system. After 10 days, corneas were harvested, lysed and subjected to Luminex for detection of cytokines.

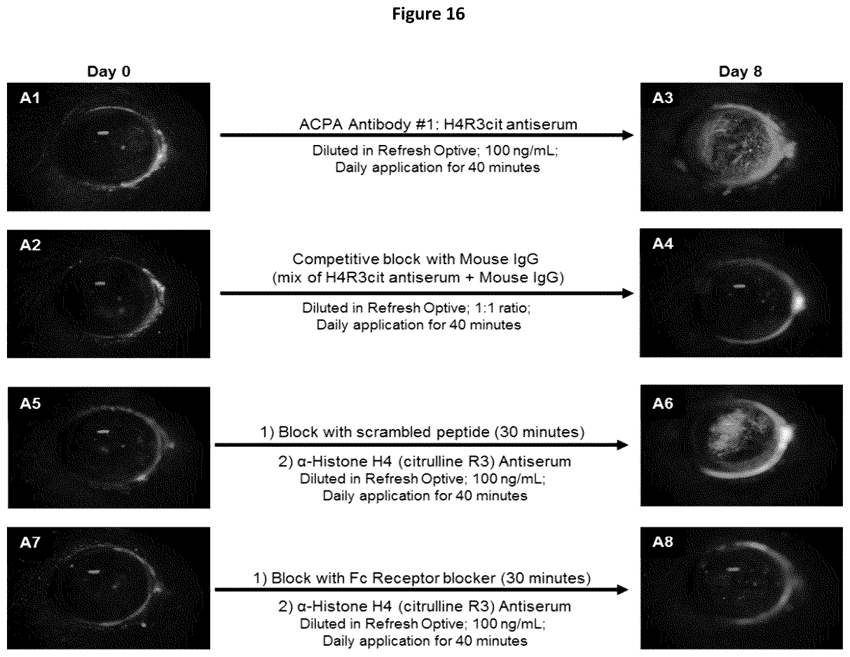

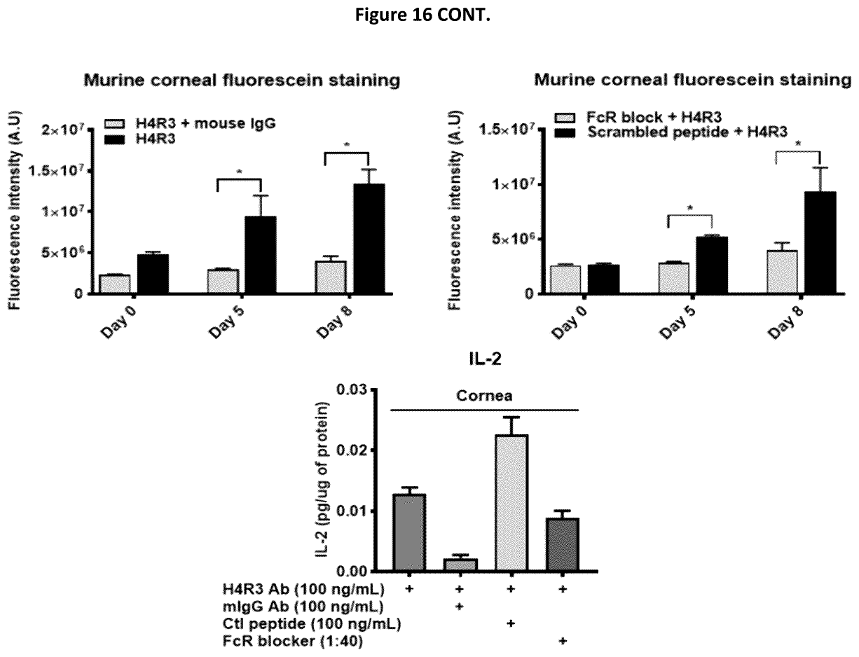

As shown in FIG. 16, both strategies significantly reduced ocular surface disease due to H4R3cit. An analysis of cytokines expressed in the cornea revealed that IL-2 was significantly increased in ACPA condition, but not in controls (FIG. 16). Blocking Fc receptor with peptide blocker reproduced the reverse effect of H4R3cit antiserum. Corneas were collected at the end of experiment, lysed, and run for cytokine profile. IL-2 level was significantly decreased in both competitive and peptide blocked mouse cornea than ones were not blocked. In summary, Mouse IgG competes with ACPA antibodies for binding to Fc receptors, and Fc receptor blocker blocks all Fc receptors. This competitive and peptide blocking abrogated pathological effect of ACPA on the cornea, suggesting that using methodologies that prevent the interaction of ACPA antibodies with Fc receptors, may be a potential therapeutic strategy in treating citrullination related eye diseases.

### Example 9: ACPA-H4R3Cit Induces NETosis In Vitro

To determine whether ACPA can induce NETosis in vitro, peripheral blood was collected by venipuncture in BD vacutainer sodium heparin tubes (BD Biosciences, #367878) and immediately transported to the laboratory for neutrophil isolation. Neutrophils were isolated by immunomagnetic depletion of non-target cells using MACSxpress beads (MACSxpress neutrophil isolation kit, Miltenyi Biotech, #130-104-434) according to the manufacturer's instruction. The residual erythrocytes were removed using MACSxpress erythrocyte depletion kit (Miltenyi Biotec, #130-094-183). Isolated neutrophils were resuspended with 3 mL of serum free phenol red free RPMI-1640 medium (GIBCO, #11835-030). After the measurement of cell numbers, 1.0×10⁶ cells/mL was plated in a chamber glass slide (Millipore, #PEZGS0416), and incubated with the following antibodies, along with controls: 1) RPMI, as negative control, 2) 1 nM PMA, as positive control, 3) 100 ng/ml normal rabbit serum (Abcam, ab7487), 4) 100 ng/ml H3cit antiserum (Novusbio, NB100-57135SS), 5) 100 ng/ml H4R3cit antiserum (Abcam, ab81797), 6) 100 ng/ml citrullinated Fibrinogen antibody (Cayman Chemical, 17088), 7) 100 ng/ml citrullinated Vimentin antibody (Cayman Chemical, 22054), 8) 100 ng/ml citrullinated α-Enolase antibody (Cayman Chemical, 23000), or 9) CCP antibody (Bioss, bs-1053R). The neutrophils were incubated for overnight, and stained with 1 Mm Sytox green (Molecular Probes, Invitrogen, cat. no. S7020) and 5 UM Hoechst (FisherScientific, Pittsburgh, PA, #33342). They were imaged immediately with Zeiss Observer Z1, at 20×.

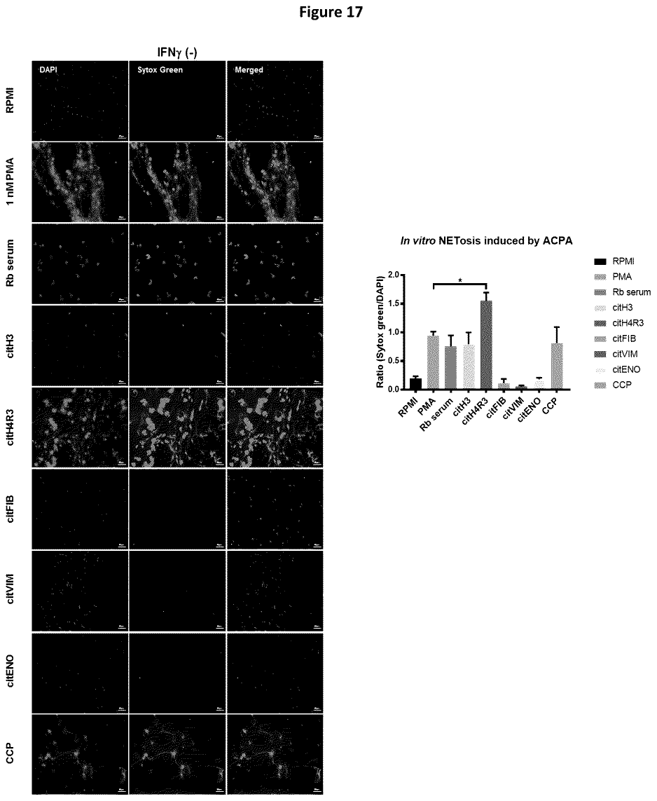

As shown in FIG. 17, the n vitro experiments using isolated human neutrophils demonstrated that ACPA-H4R3cit stimulation resulted in an significant increase in extracellular DNA strands, but not with controls (other ACPA). Only PMA stimulation, ACPA-H4R3cit stimulation and CCP ACPA stimulation produced significant extracellular DNA strands. In summary, ACPA-H4R3cit induces NETosis in vitro.

### Example 10: Determining the Cytotoxicity of Ocular Surface Immunoglobulin (OSIG) on Human Corneal Epithelial Cells Under Normal or Stressed Condition

Primary human corneal epithelial cells were used to determine the cytotoxicity OSIG on human corneal epithelial cells under normal or stressed condition Primary human corneal epithelial cells were purchased from EMD Millipore (EMD, #SCCE016). Cell were grown in EpiGROT Human Ocular Epithelia Complete MedUM (EMD, #SCMC001). A day before wound scratch, 20,000 cells/well were seeded in a 96-well ImageLock plate (Essen Bioscience, #4379) and allowed to grow 18 hours to attain monolayer confluence. The ImageLock plate is a specially-modified plate and its technology is enabled by fiducial markers on the bottom of the plate which provide points from which image locations can be accurately referenced. Wound scratch (700-800 μm wide) made with an IncuCyte 96-pin wound maker (Essen Bioscience, #4493). After scratching, cells were washed twice with 100 μL of phenol red free RPMI-1640 medium (Gibco #11835030). For OSIG (Flebogamma 5% DIF) dose-dependent experiment, medium was replaced with 200 μL of the following conditioned medium: (1) EpiGRO, (2) 4 mg/mL OSIG, or (3) 8 mg/mL OSIG. OSIG dilutions were made in EpiGRO. Plates were incubated in IncuCyte Zoom live cell analysis system (Essen Bioscience). Images were captured for every 3 hours. The relative wound density (%) was determined for 69 hours with IncuCyte Zoom software. This metric relies on measuring the spatial cell density in the wound area relative to the spatial cell density outside of the wound area at every time point. It is designed to be 0% at t=0, and 100% when the cell density inside the wound is the same as the cell density outside the initial wound. It does not rely on finding cell boundaries. Cytotoxicity of HCE-T cells was determined by LDH (lactose dehydrogenase) cytotoxicity assay (Thermo Scientific, #88954). Cell culture supernatants were collected and 50 μL of supernatant was mixed 50 μL of reaction mix and incubated for 30 min and the mix was transferred to a 96-well flat bottom plate. The absorbance at wavelengths (490-680 nm) measured with a Cytation5 plate reader.

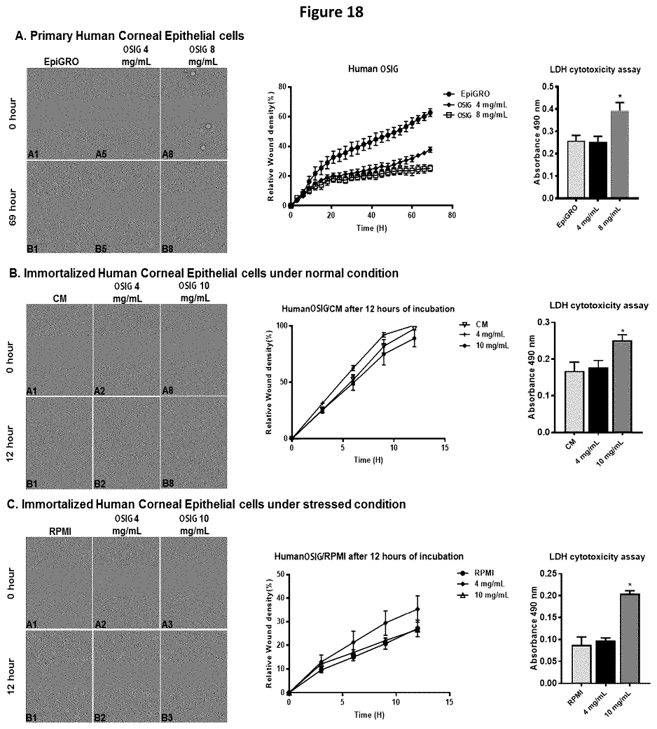

Human corneal epithelium (HCE-T) cell line was used for epithelial scratch assay and this cell line is SV40-Adeno vector transformed human cornea cells (RIKEN Cell Bank RCB2280, Tsukuba, Japan). Cells were grown in DMEM medium (GIBCO, #11965-092) supplemented with 10% FBS (Invitrogen, #26140-079) and 1% antibiotic and antimycotic solution that contains 10,000 units/mL of penicillin, 10,000 μg/mL of streptomycin, and 25 μg/mL of Gibco Amphotericin B (Thermo Fisher Scientific, #15240062) and incubated in 37° C. tissue culture incubator supplied with 5% CO2. A day before wound scratch, 30,000 cells/well were seeded in a 96-well ImageLock plate (Essen Bioscience, #4379) and allowed to grow 18 hours to attain monolayer confluence. The ImageLock plate is a specially-modified plate and its technology is enabled by fiducial markers on the bottom of the plate which provide points from which image locations can be accurately referenced. Wound scratch (700-800 μm wide) made with an IncuCyte 96-pin wound maker (Essen Bioscience, #4493). After scratching, cells were washed twice with 100 μL of phenol red free RPMI-1640 medium (Gibco #11835030). For OSIG (Flebogamma 5% DIF) dose-dependent experiment, medium was replaced with 200 μL of the following conditioned medium: (1) under normal condition (Fig. B): (i) complete medium (CM); (ii) 4 mg/mL OSIG or (iii) 10 mg/mL OSIG, (2) stressed condition (Fig. C): (i) RPMI; (ii) 4 mg/mL OSIG or (iii) 10 mg/mL. Plates were incubated in IncuCyte Zoom live cell analysis system (Essen Bioscience). Images were captured for every 3 hours. The relative wound density (%) was determined for 12 hours with IncuCyte Zoom software. This metric relies on measuring the spatial cell density in the wound area relative to the spatial cell density outside of the wound area at every time point. It is designed to be 0% at t=0, and 100% when the cell density inside the wound is the same as the cell density outside the initial wound. It does not rely on finding cell boundaries. Cytotoxicity of HCE-T cells was determined by LDH (lactose dehydrogenase) cytotoxicity assay (Thermo Scientific, #88954). Cell culture supernatants were collected and 50 μL of supernatant was mixed 50 μL of reaction mix and incubated for 30 min and the mix was transferred to a 96-well flat bottom plate. The absorbance at wavelengths (490-680 nm) measured with a Cytation5 plate reader. 10 mg/mL OSIG showed toxicity in all three conditions, whereas 4 mg/mL was not toxic, which could serve as a working concentration (effective and not toxic). As shown in FIG. 18, the toxicity did not change whether the cells were under normal or stressed condition. In summary, 4 mg/mL OSIG is the highest concentration with no toxicity.

### Example 11: ACPA Response on the Ocular Surface is an Active I Ocular Antibody Production Response

The number of patients with ocular surface disease which also have high ACPA in tear fluid despite not having these ACPA antibodies in serum and being rheumatoid factor negative was investigated. 49% of 166 patients were cyclic citrullinated peptide (CCP) negative in serum but were ACPA positive in tear fluid. 69% of these patients were rheumatoid factor negative. This suggests that the ACPA response on the ocular surface was an active local antibody production response and not a passive extravasation of ACPA from serum and it does not require a concomitant systemic immune disorder.

| | Serum CCP+ | Serum CCP− | |
|---|---|---|---|
| Tear Fluid ACPA+ | 19 | 81 | RF+ 22 |
| | Mean ACPA: 93 ± 78 SD | Mean ACPA: 28 ± 36 SD | RF− 48 |
| | Median ACPA: 86 | Median ACPA: 12.6 | |
| Tear Fluid ACPA− | 2 | 64 | RF+ 18 |
| | | | RF− 32 |

### Example 12: Examples of Patients Ary Embodiment of Eye with High Autoantibodies (ACPA) in Tear Fluid and Severe Ocular Surface Disease

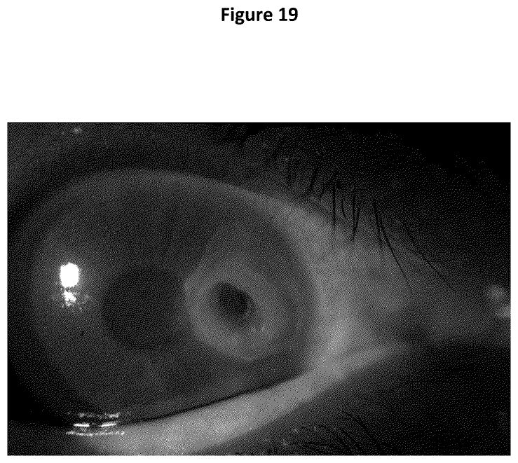

The first patient was a 38 year old female with Sjogren syndrome. As shown in FIG. 19, the patient had high autoantibodies (ACPA) in the tear fluid had a corneal melt in the right eye. Despite treatment with steroid eye drops, serum tears and other conventional DED therapies, the patient continued to remain severely symptomatic. This case demonstrates an association between high autoantibody levels and severe ocular surface disease.

### APCA in Tear Fluid Washings (Normal Levels are <5.0)

| | June 2018 | October 2018 |
|---|---|---|
| OD: | 121.8 | 104.2 |
| OS: | 121.4 | 142.6 |

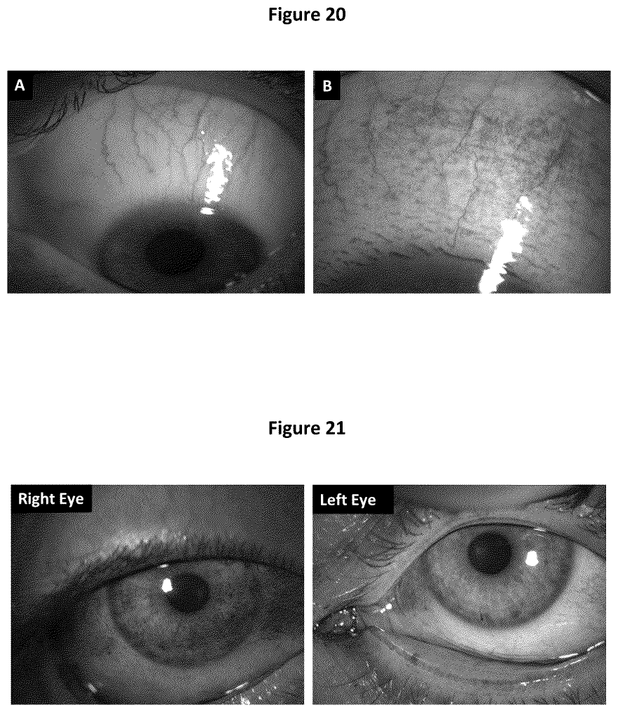

The second patient is a 39 year old female was seen in May 2018 with severe ocular discomfort. Examination revealed superior limbic keratoconjunctivitis (SLK) 3+ in both eyes. She was started on Methylprednisolone eye drops. On follow up in June 2018, symptoms of ocular discomfort still persisted despite treatment. ACPA levels in tear fluid were very high. Serum was negative for ACPA and Rheumatoid arthritis. Subsequent ACPA levels measured on July 2018 were also high and the patient still had severe symptoms. On October 2018 exam, ACPA levels reduced, although still high and the patient's symptoms were somewhat reduced. As shown in FIG. 20, this case demonstrates that autoantibodies (ACPA) were present over the eye even though there was absence of autoantibodies in the blood and there was no systemic autoimmune diseases like rheumatoid arthritis This s suggested that the autoantibodies were produced locally in the eye tissues.

### APCA in Tear Fluid Washings

| | June 2018 | July 2018 | October 2018 |
|---|---|---|---|
| OD: | 151.9 | 174.3 | 56.4 |
| OS: | 164.8 | 174.3 | 50.6 |

A third patient was a 47 year old female were asymmetric eye disease. This patient was seen on February 2017 with severe dry eye disease and ocular discomfort. Examination revealed that right eye had more severe ocular surface disease than left eye. Right eye tear production was much less than left eye (Schirmer I was 1 mm in right eye but 12 mm in left eye). As shown in FIG. 21, right corneal staining was 8/15 and left eye was 2/15. Right eye conjunctival staining was 6/6 and left eye was 3/6. ACPA in tear fluid was high in only the right eye but not in the left eye. The Eye with more severe ocular surface disease has higher autoantibody (ACPA) levels in tear fluid. This case demonstrates that higher ACPA levels correlate with more severe eye disease.

### Example 13: Additional Patient Case Studies (Case Studies 1-6)

In the following case studies, the patients were administered commercially available IVIG (Flebogamma 10%) which was formulated to represent OSIG. The commercially available IVIG (10%) was diluted with the ophthalmic excipient, NaCl solution, so that the final concentration was 4 mg/ml IgG (0.4%) with a pH of at least 6.0. An eye dropper was used to administer the resulting 0.4% IgG formulation (referred to herein as “OSIG”) to the patients as eye drops. OSIG is not currently commercially available and therefore it was fabricated using the commercially available IVIG (Flebogamma 10%), but the OSIG formulation may be fabricated using any commercially available IgG or pooled plasma-derived pooled human immunoglobulin G. These case studies provide clinically significant results that relate to the ophthalmic formulations provided herein.

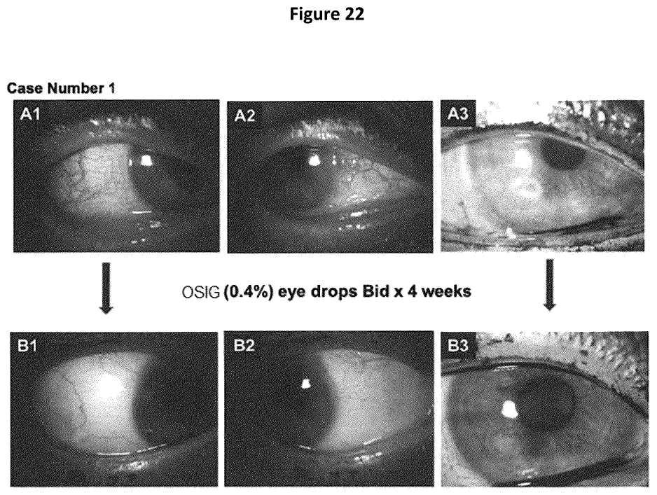

Case study 1 was a 56 year old female with severe tear deficiency and severe ocular surface disease due to ocular Graft-VS-Host Disease (FIG. 22). OSDI was 77.7 and corneal staining was 6/15 in the right eye. VBR showed ocular redness at 70 in right eye. A corneal scar was present without any corneal neovascularization. OSIG eye drops 0.4% twice a day was started. Patient reported significant reduction in ocular discomfort after two weeks of treatment (OSDI reduced to 30.5), significant reduction in corneal staining (2/15) and significant reduction in VBR (50). In the subsequent two weeks of treatment, VBR reduced to 30 and corneal staining reduced to 1/15. Extracellular DNA in tear fluid was 56 μg/mL In the right eye and after OSIG treatment reduced to 12.3 μg/mL. In this example, OSIG eye drops reduced the signs and symptoms of severe dry eye as well as reduced inflammatory biomarker in tear fluid.

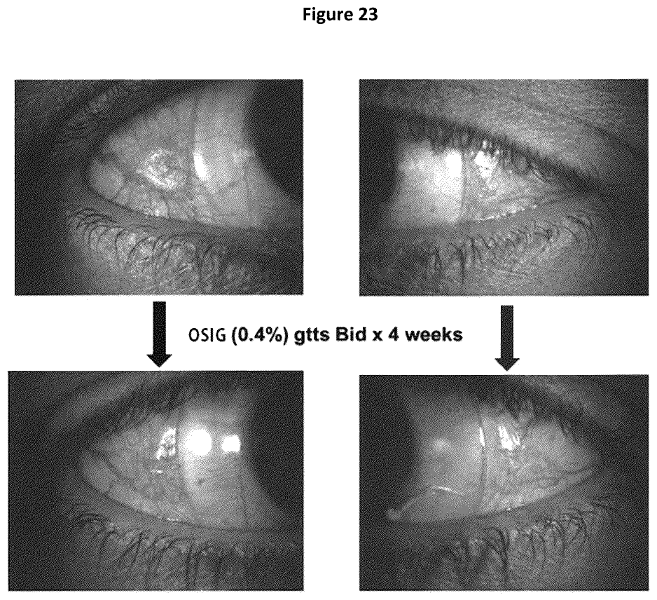

Case study 2 was a 31 year old female with severe tear deficiency and severe ocular surface disease due to ocular Graft-VS-Host Disease (FIG. 23). The patient had severe conjunctival keratinization outside the PROSE contact lens coverage area but without any corneal neovascularization and had severe symptoms of ocular discomfort. OSIG eye drops 0.4% twice a day was started. After one month of treatment, the conjunctival keratinization reduced and the patient reported “much improved” subjective ocular symptoms. In this example, OSIG eye drops reduced keratinization due to severe dry eye as well as significantly reduced ocular discomfort.

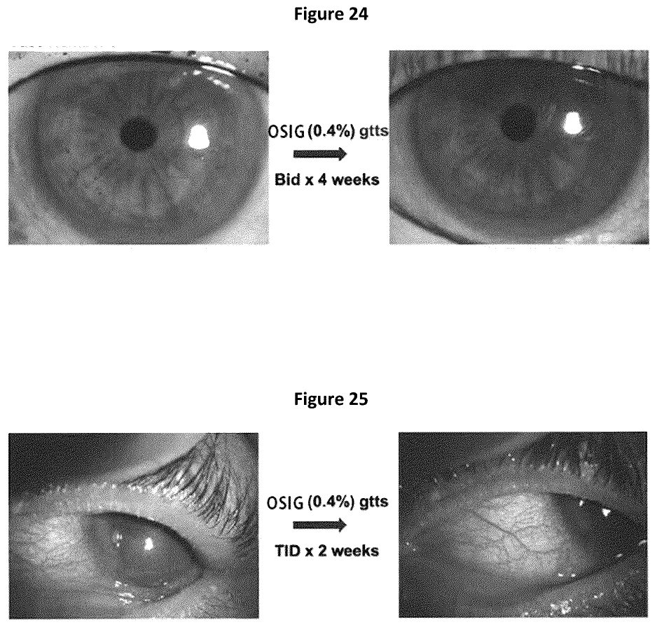

Case study 3 was a 74 year old male with neurotropic keratitis due to history of prior LASIK eye surgery in the left eye (FIG. 24). Cornea showed staining of 2/15 in the left eye and the patient reported ocular discomfort intensity of 4/10. Corneas showed faint scar at the edge of the LASIK flap without any corneal neovascularization. Overall the examination was consistent with the diagnosis of symptom sign disconnect (Discordant DED). OSIG eye drops 0.4% twice a day was started. After one month of treatment, patient was subjectively improved (SGA) and symptom intensity reduced to 3. The corneal staining disappeared (0/10) in the left eye. In this example, OSIG eye drops were beneficial in post-surgical complication leading to symptom-sign disconnect.

Case study 4 was a 39 year old male with severe tear deficiency and severe ocular surface disease Ocular Cicatricial Pemphigoid. Cornea had shown SPK staining of 1/15 in the right eye and 3/15 in the left eye without any corneal neovascularization. Patient reported ocular discomfort intensity of 3/10. OSIG eye drops 0.4% twice a day was started. After one month of treatment, the ocular discomfort intensity reduced to 2/10 and corneal staining disappeared in both eyes (0/10). This example demonstrates the beneficial effect of OSIG eye drops in ocular cicatricial pemphigoid.

Case study 5 was s a 33 year old with severe tear deficiency and severe symptoms of ocular discomfort with the diagnosis of symptom sign disconnect (Discordant DED). There was no corneal staining and there was no corneal neovascularization. The ocular discomfort intensity was 6/10. OSIG eye drops 0.4% twice a day was started. After one month of treatment, the ocular discomfort intensity reduced to 3/10 in the right eye and 4/10 in the left eye. This example demonstrates the beneficial effect of OSIG eye drops in reducing ocular discomfort in patients with symptom sign disconnect.

Case study 6 was a 29 year old male with tear deficiency and severe ocular surface disease due to Steven Johnsons Syndrome (FIG. 25). OSDI was 54.7 and symptom intensity was 8/10 (Intense horrible discomfort). OSIG eye drops 0.4% three times a day were started in both eyes. Patient reported significant reduction in ocular discomfort after two weeks of treatment (OSDI reduced to 14.2), and symptom intensity reduced to 0/10 (no discomfort). Light sensitivity and grittiness in the eye was present ‘all the time’ before OSIG treatment. After OSIG treatment, light sensitivity was only ‘some of the time’ and grittiness was not present at all. Ocular redness also reduced with OSIG treatment. In this example, OSIG eye drops significantly reduced the signs and symptoms of dry eye after Steven Johnson Syndrome.

1.-84. (canceled)

85. An ophthalmic composition formulated for administering topically to an ocular surface, said ophthalmic composition comprising:
(a) a pharmaceutically acceptable ophthalmic excipient; and
(b) a therapeutically effective amount of a pharmaceutically active compound comprising a pooled human immunoglobulin G (IgG),
wherein said pharmaceutically active compound is ocularly active and is capable of treating a clinical condition comprising (i) an inflammatory ocular surface or intraocular disease; or (ii) an immunological ocular surface or intraocular disease.

86. The ophthalmic composition according to claim 85, wherein an amount of said pooled human IgG ranges from about 0.01% by weight/volume (% w/v) to about 10% w/v.

87. The ophthalmic composition according to claim 86, wherein the amount of said pooled human IgG ranges from about 1% w/v to about 10% w/v.

88. The ophthalmic composition according to claim 87, wherein the amount of said pooled human IgG is about 5% w/v.

89. The ophthalmic composition according to claim 84, wherein said pooled human IgG comprises a pooled human IgG1, IgG2, IgG3, IgG4, or a combination thereof.

90. The ophthalmic composition according to claim 84 further comprising a growth factor.

91. The ophthalmic composition according to claim 84 further comprising an ocular surface regeneration promoting protein.

92. A method for treating an ocular disease or condition in a subject comprising administering to a subject in need of such a treatment a therapeutically effective amount of an ophthalmic composition of claim 84, wherein said ocular disease or condition comprises:
(i) an inflammatory ocular surface disease or condition,
(ii) an inflammatory intraocular disease or condition,
(iii) an immunological ocular surface disease or condition,
(iv) an immunological intraocular disease or condition, or
(v) a combination thereof.

93. The method according to claim 92, wherein said ocular disease or condition comprises inflammatory and/or immunological condition associated with ocular graft-versus-host disease, Steven Johnson syndrome, ocular cicatricial pemphigoid, tear deficient dry eye disease, meibomian gland disease, hordeolum, ocular rosacea, blepharitis, superior limbic keratoconjunctivitis, floppy eyelid syndrome, neurotrophic eye disease, symptom-sign disconnect, neuropathic pain, thyroid eye disease, rheumatoid arthritis-related eye disease, lupus-related eye disease, Sjogren's syndrome, Secondary Sjogren's syndrome, ocular rosacea, allergic keratoconjunctivitis, viral keratoconjunctivitis, Thygeson's keratitis, retinal gliosis, aniridia, keratitis, a postoperative/post-trauma ocular condition, peripheral ulcerative keratitis, episcleritis, scleritis, Uveitis, or glaucoma.

94. The method according to claim 93, wherein said ocular disease or condition is keratitis and said keratitis is due to (i) sterile inflammation or (ii) a viral, bacterial, or fungal infection.

95. The method according to claim 93, wherein said ocular disease or condition is postoperative/post-trauma ocular condition and said postoperative/post-trauma ocular condition comprises an ocular condition associated with post-ocular surface reconstruction surgery, antimetabolite application to eye surface, pterygium surgery, glaucoma surgery, cataract surgery, refractive surgery, keratoprosthesis surgery, or radiation or chemical or traumatic injury.

96. The method according to claim 92, wherein an amount of said pooled human IgG ranges from about 0.01% by weight/volume (% w/v) to about 10% w/v.

97. The method according to claim 96, wherein the amount of said pooled human IgG ranges from about 1% w/v to about 10% w/v.

98. The method according to claim 97, wherein the amount of said pooled human IgG is about 5% w/v.

99. An ophthalmic composition formulated for a topical administration to an ocular surface, said ophthalmic composition comprising:
(a) a pharmaceutically acceptable ophthalmic excipient; and
(b) a pooled human plasma, serum, or blood comprising from about 0.01% w/v to about 10% w/v of immunoglobulin G (IgG).

100. The ophthalmic composition according to claim 99, wherein the amount of said pooled human IgG ranges from about 1% w/v to about 10% w/v.

101. The ophthalmic composition according to claim 99, wherein said pooled human IgG comprises a pooled human IgG1, IgG2, IgG3, IgG4, or a combination thereof.

102. The ophthalmic composition according to claim 99 further comprising a growth factor.

103. An ophthalmic composition formulated for a topical administration to an ocular surface, said ophthalmic composition comprising:
(a) a pharmaceutically acceptable ophthalmic excipient; and
(b) about 5% w/v of pooled human immunoglobulin G (IgG).

104. The ophthalmic composition according to claim 103 further comprising a growth factor.
